(19) United States
(12) Patent Application Publication
(10) Pub. No.: US 2020/0393469 A1
(43) Pub. Date: Dec. 17, 2020

(54) METHOD FOR DETERMINING ELIGIBILITY OF BRAIN TUMOR PATIENT FOR TAILOR-MADE TYPE PEPTIDE VACCINE AGENT

(71) Applicant: KURUME UNIVERSITY, Fukuoka (JP)

(72) Inventors: Kyogo ITO, Miyaki-gun, Saga (JP); Shigeki SHICHIJO, Kurume-shi, Fukuoka (JP)

(73) Assignee: KURUME UNIVERSITY, Fukuoka (JP)

(21) Appl. No.: 16/613,177

(22) PCT Filed: May 16, 2018
(86) PCT No.: PCT/JP2018/018936

§ 371 Date: Nov. 13, 2019

(30) Foreign Application Priority Data
May 16, 2017 (JP) 2017-097502

(51) Int. Cl. G01N 33/68; C07K 7/06

(52) U.S. Cl. CPC G01N 33/68; G01N 2800/60; C07K 7/06

(57) ABSTRACT

Provided are a method for determining whether a subject suffering from a brain tumor is an eligible person for a tailor-made peptide vaccine composition including at least one peptide antigen, a kit used in the aforesaid determination method, and a method for treating a subject suffering from a brain tumor by administering a tailor-made peptide vaccine composition including at least one peptide antigen.

### TECHNICAL FIELD

The present invention relates to a method for determining eligibility of a brain tumor patient for a tailor-made peptide vaccine composition.

### BACKGROUND

Cancer immunotherapies are generally and roughly divided into passive immunotherapies and active immunotherapies. The passive immunotherapies include antibody therapies that administer an inhibitory antibody against a molecule involved in proliferation of cancer cells. The active immunotherapies include vaccine therapies that inoculate a patient with an antigenic peptide.

An anti PD-1 antibody for an antibody therapy has been marketed as a lung cancer therapeutic drug. However, the PD-1 antibody has been found to be effective only in a part of the subjects (Non-Patent Literature 1).

Cancer vaccine therapies using a peptide vaccine composition have also been known to be ineffective in some cases when the peptide vaccine composition includes only a single peptide derived from a single cancer antigen. Cancer vaccine therapies using a tailor-made (made-to-order or personalized) peptide vaccine composition, including a plurality of peptide vaccines, have been recently available. The plurality of peptide vaccines are selected from a peptide library derived from a wide variety of cancer antigens based on an evaluation in advance of immunoreactivities of a subject to the peptide vaccines.

Personalized and precision medicine has recently attracted attention. It has been recognized that development of new technologies contributing to precision medicine is important in view of medical economics.

### CITATION LIST

Non-Patent Literature 1: Mariacarmela Santarpia, et al., Programmed cell death protein-1/programmed cell death ligand-1 pathway inhibition and predictive biomarkers: understanding transforming growth factor-beta role, Transl Lung Cancer Res. 2015 Dec. 4 (6): 728-42.

### SUMMARY

It is advantageous if eligibility of a subject for a tailor-made peptide vaccine composition can be determined before preparation or administration of the tailor-made peptide vaccine composition because a subject who was determined as being ineligible will have an opportunity to receive another therapy. In view of medical economics, it is also advantageous because the cost involved in the preparation or administration of the peptide vaccine composition can save. At the same time, a subject determined as being eligible can receive cancer immunotherapy using the peptide vaccine composition that is highly possibly effective. Accordingly, in the field of vaccine therapy using a tailor-made peptide vaccine composition, it has been desired to develop a method for determining in advance the eligibility of a subject for a peptide vaccine composition to be prepared.

The present inventors conducted phase III double-blind comparative test for HLA-A24-positive patients suffering from glioma that was resistant to a temozolomide treatment with use of a tailor-made peptide vaccine composition. The inventors evaluated the level of granulocyte-macrophage colony-stimulating factor (GM-CSF) in blood from the subjects who were assessed as being in performance status (PS) of grades 0 to 2 according to Eastern Cooperative Oncology Group (ECOG), and further evaluated either one or both of immunoreactivity to SART2 peptide and the blood level of Monocyte Chemoattractant Protein-1 (MCP-1) of the subjects. As a result, the inventors found that the eligibility of each of the subjects for the tailor-made peptide vaccine composition can be determined in advance. Based on the findings, the present invention has been accomplished.

### Solution to Problem

The present invention provides a method for determining whether a subject suffering from a brain tumor is an eligible person for a tailor-made peptide vaccine composition including at least one peptide antigen; a kit for use in the determination method; and a method for treating a subject suffering from a brain tumor by administering a tailor-made peptide vaccine composition including at least one peptide antigen. These are described below.

An aspect of the present invention relates to a method for determining whether a subject suffering from a brain tumor is an eligible person for a tailor-made peptide vaccine composition including at least one peptide antigen, comprising the steps of evaluating a risk of the subject with respect to the peptide vaccine composition; and determining whether the subject is an eligible person for the peptide vaccine composition based on the evaluation, wherein the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin.

An aspect of the present invention relates to a kit for use in the above-described determination method, containing a reagent for measuring at least one prognostic factor selected from the group consisting of GM-CSF, at least one SART2, MCP-1, VEGF, IL-6, IL-7, IL-10, IL-17, IL-1RA, CCL4, and haptoglobin.

An aspect of the present invention relates to a method for treating a subject suffering from a brain tumor by administering a tailor-made peptide vaccine composition including at least one peptide antigen, wherein the subject is a person who is determined as an eligible person for the peptide vaccine composition based on an evaluation on a risk of the subject with regard to the peptide vaccine composition; and the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin.

### Technical Effects

According to the determination method of the present invention, eligibility of a brain tumor patient for a tailor-made peptide vaccine composition that has been selected for the patient can be determined before the treatment with the tailor-made peptide vaccine composition starts. When the determination result is an “ineligible subject”, the patient will have an opportunity to receive another therapy. Further, the cost involved in preparation or administration of the peptide vaccine composition can reduce, which is an additional advantage in light of medical economics. On the other hand, when the determination result is an “eligible subject”, the patient will have an opportunity to receive a cancer immunotherapy using the peptide vaccine composition with a reduced risk of unsuccessful response.

### BRIEF DESCRIPTION OF DRAWINGS

FIG. 1 is a line graph showing the survival rates of test subjects who participated in a clinical trial. The solid line represents the survival rate of the test subjects in a group with administration of an active drug. The broken line represents the survival rate of the test subjects in a group with administration of a placebo. The vertical axis represents the survival rate. The horizontal axis represents the number of days elapsed from the initial administration of a study product.

FIG. 2 is a line graph showing the survival rates of the test subjects. The solid line represents the survival rate of the test subjects having a PS of grade 0 to 2. The broken line represents the survival rate of the test subjects having a PS of grade 3. The vertical axis represents the survival rate. The horizontal axis represents the number of days elapsed from the initial administration of the study product.

FIG. 3 shows line graphs showing the survival rates of the test subjects. The line graph (a) shows the survival rates of the test subjects for whom the SART2 peptide was not selected (SART2−) as a component of the active drug. The line graph (b) shows the survival rates of the test subjects for whom the SART2 peptide was selected as a component of the active drug (SART2+). The solid line represents the survival rate of an active-drug group. The broken line represents the survival rate of a placebo group. The vertical axes represent the survival rate. The horizontal axes represent the number of days elapsed from the initial administration of the study product.

FIG. 4 is a diagram schematically showing results obtained by subjecting the clinical trial results of the test subjects having a PS of grade 0 to 2 to subgroup analysis based on whether the SART2 peptide was selected as a component of the active drug (SART2+) or not (SART2−).

FIG. 5 is a series of bar graphs showing blood GM-CSF concentrations of patients in the clinical cases before administration of the active drug. The bar graph (a) shows blood GM-CSF concentrations of glioma patients, (b) shows those of ureteral cancer patients, (c) shows those of bladder cancer patients, (d) shows those of esophageal cancer patients, (e) shows those of stomach cancer patients, and (f) shows those of biliary tract cancer patients. The series of bar graphs shows that the glioma patients were significantly high blood CM-CSF concentrations compared to the others. The vertical axes represent the blood GM-CSF concentration (pg/mL) of individual patients. The horizontal axes represent the clinical cases arranged in the ascending order of GM-CSF concentration from the left.

FIG. 6 shows line graphs showing the survival rates of the test subjects. The line graph (a) shows the survival rates when the blood GM-CSF concentration was less than 0.9 pg/mL. The line graph (b) shows the survival rates when the blood GM-CSF concentration was not less than 0.9 pg/mL. The solid line represents the active-drug group. The broken line represents the placebo group. The vertical axes represent the survival rate. The horizontal axes represent the number of days elapsed from the initial administration of the study product.

FIG. 7 is a diagram schematically showing results obtained by subjecting the clinical trial results of the test subjects having a PS of grade 0 to 2 to a subgroup analysis based on the blood GM-CSF concentrations of the test subjects before administration of the active drug or the placebo.

FIG. 8 shows line graphs showing the survival rates of the test subjects. The line graph (a) shows the survival rates when the blood GM-CSF concentration was less than 0.9 pg/mL or the SART2 peptide was not selected as a component of the active drug (SART2−). The line graph (b) shows the survival rates when the blood GM-CSF concentration was not less than 0.9 pg/mL and the SART2 peptide was selected as a component of the active drug (SART2+). The solid line represents the survival rate of an active-drug group. The broken line represents the survival rate of the placebo group. The vertical axes represent the survival rate. The horizontal axes represent the number of days elapsed from the initial administration of the study product.

FIG. 9 is a diagram schematically showing results obtained by subjecting the clinical trial results of the test subjects having a PS of grade 0 to 2 to a subgroup analysis based on two factors, SART2 and GM-CSF.

FIG. 10 shows line graphs showing the survival rates of the test subjects. The line graph (a) shows the survival rates when the blood GM-CSF concentration was less than 0.9 pg/mL or the SART2 peptide was not selected as a component of the active drug (SART2−). The line graph (b) shows the survival rates when the blood GM-CSF concentration was not less than 0.9 pg/mL and the SART2 peptide was selected as a component of the active drug (SART2+). The broken line represents the survival rates of the test subjects when an antibody amount ratio before and after the administration of the active drug was less than 2. The solid line represents the survival rates of the test subjects when the antibody amount ratio was two or more. The vertical axes represent the survival rate. The horizontal axes represent the number of days elapsed from the initial administration of the study product.

FIG. 11 is a bar graph showing the blood MCP-1 concentrations of the test subjects. The blood MCP-1 concentrations [1] and [2] were of the placebo groups before the administration of the placebo shown in FIG. 9. The blood MCP-1 concentrations [3] and [4] were of the active-drug groups before the administration of the active drug shown in FIG. 9. The vertical axes represent blood MCP-1 concentrations (pg/mL) of the individual test subjects. The horizontal axes represent the clinical cases arranged in the ascending order of the MCP-1 concentrations from the left for each of the groups [1] to [4].

FIG. 12 shows line graphs showing the survival rates of the test subjects. The line graph (a) shows the survival rates in the active-drug group. The line graph (b) shows the survival rates in the placebo group. The solid line shows the survival rates when the blood MCP-1 concentration was not less than 100 pg/mL. The broken line shows the survival rates when the blood MCP-1 concentration was less than 100 pg/mL. The vertical axes represent the survival rate. The horizontal axes represent the number of days elapsed from the initial date of the active drug or the placebo administration.

FIG. 13 is a diagram schematically showing results obtained by subjecting the clinical trial results of the test subjects having a PS of grade 0 to 2 to a subgroup analysis based on the blood MCP-1 concentrations of the test subjects before administration of the active drug or the placebo.

FIG. 14 shows line graphs showing the survival rates. The line graphs (a) and (b) show the survival rates of 15 test subjects who have the blood MCP-1 concentration of less than 100 pg/mL before administration of the active drug. The line graph (a) shows a solid line that represents the survival rates of the test subjects having a GM-CSF concentration of not less than 0.9 pg/mL and a broken line that represents the survival rates of the test subjects having a GM-CSF concentration of less than 0.9 pg/mL. The line graph (b) shows a solid line that represents the survival rates of the test subjects for whom the SART2 peptide was not selected as a component as the active drug (SART2−) and a broken line that represents the survival rates of the test subjects for whom the SART2 peptide was selected (SART2+). The line graphs (c) and (d) show the survival rates of 32 test subjects who have the blood MCP-1 concentration of not less than 100 pg/mL before the administration of the active drug. The line graph (c) shows a solid line that represents the survival rates of the test subjects who have the GM-CSF concentration of not less than 0.9 pg/mL and a broken line that represents the survival rates of the test subjects who have the GM-CSF concentration of less than 0.9 pg/mL. The line graph (d) shows a solid line that represents the survival rates of the test subjects for whom the SART2 peptide was not selected as a component as the active drug (SART2−) and a broken line that represents the survival rates of the test subjects for whom the SART2 peptide was selected (SART2+). The vertical axes represent the survival rate. The horizontal axes represent the number of days elapsed from the initial administration of the active drug.

FIG. 15 is a scatter graph showing MCP-1 concentrations (beyond 700 pg/mL) of the test subjects versus their survival time. A first group is the active-drug group consisting of the subjects whose GM-CSF concentration was less than 0.9 pg/mL or for whom the SART2 peptide was not selected as a component of the active drug (SART2−). A second group is the placebo group consisting of the subjects whose GM-CSF concentration was less than 0.9 pg/mL or for whom the SART2 peptide was not selected (SART2−). A third group is the active-drug group consisting of the subjects whose GM-CSF concentration was not less than 0.9 pg/mL and for whom the SART2 peptide was selected as a component of the active drug (SART2+). A fourth group is the active-drug group consisting of the subjects whose GM-CSF concentration was not less than 0.9 pg/mL and for whom the SART2 peptide was selected (SART2+). The vertical axis represents survival time (days). The horizontal axis represents MCP-1 concentration (pg/mL).

FIG. 16 is a line graph showing the survival rates of the test subjects whose MCP-1 felt within the range of 100 pg/mL to 700 pg/mL or whose GM-CSF concentration was less than 0.9 pg/mL. The solid line represents the active-drug group. The broken line represents the placebo group. The vertical axis represents the survival rate. The horizontal axis represents the number of days elapsed from the initial administration of the study product.

FIG. 17 is a line graph showing the survival rates of the test subjects (9 subjects) whose blood MCP-1 concentration before administration of the active drug was less than 100 pg/mL and for whom the SART2 peptide was selected as a component as the active drug (SART2+). The solid line represents the survival rates of the test subjects having a GM-CSF concentration of less than 0.9 g/mL. The broken line represents the survival rates of the test subjects having a GM-CSF concentration of not less than 0.9 g/mL. The vertical axis represents the survival rate. The horizontal axis represents the number of days elapsed from the initial administration of the active drug.

FIG. 18 is a line graph showing the survival rates of the test subjects having a blood CCL4 concentration before administration of the active drug. The test subjects having the blood CCL4 concentration of less than the median of the active-drug group were 27. The test subjects having a blood CCL4 concentration of not less than the median of the active-drug group were 26. The vertical axis represents the survival rate. The horizontal axis represents the time (months) elapsed from the initial administration of the active drug.

FIG. 19(a) is a bar graph showing the relationship between the blood CCL2 (MCP-1) concentration and the overall survival in the active-drug group. FIG. 19(b) is a line graph showing the survival rates of the test subjects having a blood CCL2 concentration before administration of the active drug. The test subjects having a blood CCL2 concentration of very low or high (high/low) were 12. The test subjects having a blood CCL2 concentration of intermediate (im) were 41. In FIG. 19(a), the vertical axis represents the overall survival time (months). The horizontal axis represents test subjects arranged in the ascending order of the CCL2 level from the left. SASR2-93 (−) stands for the test subject for whom SART2-93 was not selected as a component of the peptide vaccine. In FIG. 19(b), the vertical axis represents the survival rate. The horizontal axis represents the time (months) elapsed from the initial administration of the active drug.

FIG. 20(a) is a bar graph showing the relationship between the blood VEGF concentration and the overall survival in the active-drug group. FIG. 20(b) is a line graph showing the survival rates of the test subjects having a blood VEGF concentration before administration of the active drug. The test subjects having a blood VEGF concentration of very low or high (high/low) were 12. The test subjects having a blood VEGF concentration of intermediate (im) were 41. In FIG. 20(a), the vertical axis represents the overall survival time (months). The horizontal axis represents test subjects arranged in the ascending order of the VEGF level from the left. SASR2-93(−) stands for the test subjects for whom SART2-93 was not selected as a component of the peptide vaccine. In FIG. 20(b), the vertical axis represents the survival rate. The horizontal axis represents the time (months) elapsed from the initial administration of the active drug.

FIG. 21(a) is a bar graph showing the relationship between the blood haptoglobin (Hp) concentration and the overall survival in the active-drug group. FIG. 21(b) is a line graph showing the survival rates of the test subjects having a blood Hp concentration before administration of the active drug. The test subjects having a Hp concentration of very low or high (high/low) were 8. The test subjects having a blood Hp concentration of intermediate (im) were 45. In FIG. 21(a), the vertical axis represents the overall survival time (months). The horizontal axis represents test subjects arranged in the ascending order of the Hp level from the left. SASR2-93(−) stands for the test subjects for whom SART2-93 was not selected as a component of the peptide vaccine. In FIG. 21(b), the vertical axis represents the survival rate. The horizontal axis represents the time (months) elapsed from the initial administration of the active drug.

FIG. 22(a) is a bar graph showing the relationship between the blood GM-CSF concentration and the overall survival in the active-drug group. FIG. 22(b) is a line graph showing the survival rates of the test subjects having a blood GM-CSF concentration before administration of the active drug. The test subjects having very high (high) were 5. The test subjects having a blood GM-CSF concentration of not high were 48. In FIG. 22(a), the vertical axis represents the overall survival time (months). The horizontal axis represents test subjects arranged in the ascending order of the GM-CSF level from the left. SASR2-93(−) stands for the test subjects for whom SART2-93 was not selected as a component of the peptide vaccine. In FIG. 22(b), the vertical axis represents the survival rate. The horizontal axis represents the time (months) elapsed from the initial administration of the active drug.

FIG. 23 is a line graph showing the survival rates of the test subjects having a blood IL-15 concentration before administration of the placebo. The test subjects having a blood IL-15 concentration of not less than the median of the placebo group were 17. The test subjects having a blood IL-15 concentration of less than the median value were 13. The vertical axis represents the survival rate. The horizontal axis represents the time (months) elapsed from the initial administration of the active drug.

FIG. 24(a) is a bar graph showing the relationship between the blood IL-6 concentration and the overall survival in the placebo group. FIG. 24(b) is a line graph showing the survival rates of the test subjects having a blood IL-6 concentration before administration of the placebo. The test subjects having a blood IL-6 concentration of very low or high (high/low) were 8. The test subjects having a blood IL-6 concentration of intermediate (im) were 22. In FIG. 24(a), the vertical axis represents the overall survival time (months). The horizontal axis represents test subjects arranged in the ascending order of the IL-6 level from the left. SASR2-93(−) stands for the test subjects for whom SART2-93 was not selected as a component of the peptide vaccine. In FIG. 24(b), the vertical axis represents the survival rate. The horizontal axis represents the time (months) elapsed from the initial administration of the active drug.

FIG. 25(a) is a bar graph showing the relationship between the blood CCL2 concentration and the overall survival in the placebo group. FIG. 25(b) is a line graph showing the survival rates of the test subjects having a blood CCL2 concentration before administration of the placebo. The test subjects having a blood CCL2 concentration of very low or high (high/low) were 7. The test subjects having a blood CCL2 concentration of intermediate (im) were 23.

FIG. 25(c) is a line graph showing the survival rates of the test subjects having a blood CCL2 concentration before administration of the placebo. The test subjects having a blood CCL2 concentration of very low or high (high/low) were 7 in the placebo group (Best Supportive Care: BSC) and were 12 in the active-drug group. In FIG. 25(a), the vertical axis represents the overall survival time (months). The horizontal axis represents test subjects arranged in the ascending order of the CCL2 level from the left. In FIG. 25(b), the vertical axis represents the survival rate. The horizontal axis represents the time (months) elapsed from the initial administration of the placebo. In FIG. 25(c), the vertical axis represents the survival rate. The horizontal axis represents the time (months) elapsed from the initial administration of the active drug.

FIG. 26 is a series of graphs regarding of 37 test subjects inoculated with a tailor-made peptide vaccine (personalized peptide vaccine; PPV) and 21 test subjects who received Best Supportive Care (BSC). FIG. 26(a) is a bar graph showing the ratio of CD11b⁺CD14⁺HLA-DR^{low} immunosuppressive monocytes of lymphocytes of the blood of the test subjects before and after inoculation with the PPV or the placebo. FIG. 26(b) is a bar graph showing the ratio of CD3⁺CD4⁺CD45RA⁻T cells in the lymphocytes of the blood of the test subjects before and after inoculation with the PPV or the placebo. FIG. 26(c) is a bar graph showing the ratio of CD4⁺CD25⁺FoxP3⁺ cell (Treg) in the lymphocytes of the blood of the test subjects before and after inoculation with the PPV or the placebo. In FIG. 26(a) to FIG. 26(c), the vertical axis represents the ratio of predetermined lymphocytes to whole lymphocytes. The horizontal axis represents pre-inoculation (pre) and post-inoculation (post) of the PPV or the placebo. FIG. 26(d) is a line graph showing the correlation between the ratio of CD11b⁺CD14⁺HLA-DR^{low} immunosuppressive monocytes and the overall survival of the test subjects (n=45) received with the PPV. FIG. 26(e) is a line graph showing the correlation between the ratio of CD11b⁺CD14⁺HLA-DR⁻ immunosuppressive monocytes and the overall survival of the test subjects. In FIG. 26(d) and FIG. 26(e), the vertical axis represents the survival rate. The horizontal axis represents the time (months) elapsed from the initial administration of the study product.

### DESCRIPTION OF EMBODIMENTS

In the specification, the “brain tumor” refers to a tumor that develops within the cranium intracranial and also called as an intracranial tumor. Examples of the brain tumor include glial tumor, meningioma, pituitary adenoma and schwannoma. The “glial tumor”, which is a generic name of tumors developed from the neuroectoderm tissue of the brain parenchyma, is the most frequent brain tumor that accounts for 30 to 40% of the primary intracranial tumors. The “meningioma”, which accounts for about 15% of the brain tumors, is developed from arachnoid villus cells of arachnoid granules and known to frequently occur in adults. The “pituitary adenoma”, which accounts for about 15% of the brain tumors, refers to a benign tumor developed from the anterior pituitary cells and is known to frequently occur in adults (20 to 50 years old). The “schwannoma” refers to a benign lesion surrounded by fibrous capsule. In an embodiment, brain tumor is a malignant brain tumor, for example, glial tumor.

Examples of the glial tumor include, but not limited to, astrocytoma, glioma, ventricular ependymoma, oligodendroglioma and choroid plexus papilloma. The “astrocytoma”, which accounts for 20 to 30% of the whole glial tumors, refers to a benign or malignant tumor developed in the central nervous system. The “glioma”, which accounts for about 10% of the whole glial tumors, is highly malignant and known to frequently occur in the child's cerebellum. The “ventricular ependymoma”, which accounts for 5 to 8% of the glial tumors, is known to frequently occur in young people. The “oligodendroglioma”, which accounts for 5% of the glial tumors, is known to frequently occur in adult's cerebral hemisphere. The “choroid plexus papilloma” is less-frequently occurring tumor and known to occur in the child's ventricle. In an embodiment, the glial tumor is glioma. Examples of glioma include, but not limited to, glioma that is resistant to a cancer therapy such as glioma that is resistant to a temozolomide therapy. Temozolomide, which is an orally administrable anti-cancer agent, is a prodrug having an imidazotetrazine backbone.

In the specification, “HLA”, which is an abbreviation of Human Leucocyte Antigen, refers to a human major histocompatibility complex (MHC). HLAs are roughly classified into class I antigens and class II antigens. Class I antigens are further classified into class Ia antigens (HLA-A, B, C) and class Ib antigens (HLA-E, F, G). A type of HLAs may be specified in accordance with a conventional method such as serological typing, cytological typing and DNA typing. In the present invention, the type of “HLA” of a subject is determined usually before a peptide vaccine is prepared; but may be determined at any time. In an embodiment, an HLA type may be determined before, during or after preparation of a peptide vaccine, or further determined before, during or after a dosing cycle.

In the specification, “performance status (PS)” refers to a scale indicating activities of daily life. In the specification, PS may be declared by a subject by selecting a grade from the list of activity grades in consideration of his own activities of daily life, or a third party such as a medical doctor may select PS in consideration of activities of daily life of the subject. The grades for PS may be the grades proposed by an organization such as the Eastern Cooperative Oncology Group (ECOG). In an embodiment, PS is selected from the followings: grade 0 “can act without problems (send the daily life similarly before onset, without limit)”; grade 1 “walkable and can work on light duty and while sitting although physically intensive activity is prohibited (light housework, office work)”; grade 2 “walkable, able to care for oneself but unable to work (spent outside bed during 50% or more of the day time)”; grade 3 “able to care oneself but limited (spent in bed or sitting on chair during 50% or more of the day time)”; and grade 4 “completely unable to move. Completely unable to care oneself”.

In the specification, the “subject” refers to a human suffering from a brain tumor. In an embodiment, the subject is a patient suffering from a malignant brain tumor (for example, glial tumor). In another embodiment, the subject is a patient suffering from a highly malignant glial tumor (for example, glioma). In another embodiment, the subject is a patient suffering from glioma that is resistant to another therapy, for example glioma that is resistant to a temozolomide therapy. In another embodiment, the subject is a patient suffering from a malignant brain tumor (for example, glial tumor, glioma, or chemotherapy resistant/tolerant glioma (for example, glioma that is resistant to a temozolomide therapy)), having PS of grade 0 to 2. In an embodiment, the patient described above is HLA-A24 positive.

In the specification, the “peptide antigen” refers to a peptide derived from a tumor-associated antigen protein for inducing a tumor-specific immune response. Peptide antigens are roughly divided into short-chain peptide antigens and long-chain peptide antigens depending on difference in chain length. In the specification, examples of the peptide antigens include, but not limited to, a chemical synthesized peptide antigen or an isolated/purified peptide antigen from a biological sample. Examples of the method for chemically synthesizing a peptide include, but not limited to, the Fmoc method and azide method. Examples of the peptide antigen isolated/purified from a biological sample include, but not limited to, a peptide antigen produced by peptide synthesis using genetically engineering technique. In an embodiment, the peptide antigen may include either one or both of the chemically synthesized short-chain peptide antigen and long-chain peptide antigen. In another embodiment, the peptide antigen includes a chemically synthesized short-chain peptide.

In the specification, “short-chain peptide antigen” refers to an epitope peptide having a chain length sufficient to directly bind to MHC on the surface of an antigen-presenting cell and not to be incorporated. In an embodiment, a short-chain peptide antigen consists of 8 to 17 amino acid residues. In order to induce killer T cells, the peptide antigen may be 8 to 11 amino acid residues in length. In order to induce helper T cells, the peptide antigen may be 12 to 17 amino acid residues in length. In an embodiment, a short-chain peptide antigen consists of 8 to 10 amino acid residues.

In the specification, “long-chain peptide antigen” refers to a peptide having a relatively long chain length and containing one to a plurality of epitopes. The long-chain peptide antigen is usually unable to directly bind to MHC, and it is incorporated into an antigen-presenting cell and processed within the cell into epitope peptides by the function of a protease and a peptidase within an endosome and a proteasome within the cytoplasm. The long-chain peptide antigen may have a natural amino acid sequence of a tumor-associated antigen protein, or an amino acid sequence prepared by artificially linking a plurality of epitope peptides. In an embodiment, the long-chain peptide antigen consists of 20 to 80 amino acid residues and preferably 20 to 50 amino acid residues.

In the present invention, the “peptide vaccine” refers to a “tailor-made peptide vaccine”, unless otherwise specified. The tailor-made peptide vaccine is also referred to a personalized peptide vaccine (PPV) or an order-made peptide vaccine and means a peptide vaccine prepared by analyzing a tumor-associated antigen expressed in a subject to whom the peptide vaccine is to be administered or tumor specific immunoreactivity of the subject, and selecting a peptide antigen to be contained in the peptide vaccine based on the analysis results.

In an embodiment, the tailor-made peptide vaccine includes at least one peptide antigen that is selected by choosing a peptide antigen group corresponding to the HLA type of a subject from peptide antigen groups each specified for one of HLA types, and then selecting from individual peptide antigens constituting the peptide antigen group based on immunoreactivities of the subject to the individual peptide antigens. The peptide vaccine may include at least one peptide antigen selected from individual peptide antigens constituting a peptide antigen group specified for HLA-A24 based on the immunoreactivities of a subject to the individual peptide antigens.

The immunoreactivity of a subject to a peptide antigen can be determined by an antibody test or the like using a sample obtained from the subject, for example, body fluid or blood (for example, whole blood, plasma or serum). Examples of the antibody test include enzyme-linked immunosorbent assay (ELISA) and an assay using a flow cytometer or flow meter (also called as “Bead-based multiplex assays” and “fluorescent bead array”). The immunoreactivity may be determined as being positive when the value of immunoreactivity is sufficiently high compared to the quantitative value obtained in a negative control.

In an embodiment, the immunoreactivity may be measured as follows. In an embodiment, the immunoreactivity may be evaluated as being “positive” when the value of immunoreactivity is larger than a value that is a quantitative value (average value) of a placebo (containing an adjuvant but not peptide antigens) plus a value being five times as large as a standard deviation (S.D.) thereof, and larger than the integer closest to the value (average value+5×S.D.). In another embodiment, the immunoreactivity is determined by a method using flow cytometry or a flow meter. The immunoreactivity may be evaluated as being “no reactivity” or “not detected (ND)” when the level of a signal derived from a substrate (for example, fluorescence intensity FIU) is less than 10 FIU, and as being “positive” when the level is 10 FIU or more. The immunoreactivity may be evaluated as being “no reactivity” or “not detected (ND)” when the level of a signal corresponding to a peptide antigen (for example, fluorescence intensity FIU) is less than 10 FIU, and as being “positive” when the level is 10 FIU or more.

The immunoreactivity of a subject to a peptide antigen can be measured, for example, by using a substrate (for example, microtiter plate or bead) onto which a peptide antigen is immobilized so as to generate a predetermined level of a signal (for example, 1000 FIU) in response to a predetermined positive specimen (blood sample whose reactivity is known). In this case, the immunoreactivity can be determined by allowing the substrate to be in contact with a blood sample from a subject, washing the substrate, then allowing the substrate to be in contact with an anti-human antibody labeled with a marker substance (for example, fluorescence substance) (labeled secondary antibody), washing the substrate, and detecting a signal (for example, fluorescence) derived from the marker substance (for example, fluorescence substance). In this case, the immunoreactivity may be evaluated as being “no reactivity” or “not detected (ND)” when the level of a signal derived from the substrate (for example, fluorescence intensity FIU) is less than 10 FIU, and as being positive when the level is 10 FIU or more.

In an embodiment, a tailor-made peptide vaccine includes at least one peptide antigen, preferably at least two, at least three, at least four or at most four peptides selected from a peptide antigen group corresponding to the HLA type of a subject, based on immunoreactivities of the subject to peptide antigens constituting the peptide antigen group (for example, in the descending order from the highest immunoreactivity). The tailor-made peptide vaccine may include, but not limited to, two to seven peptide antigens, preferably three to six peptide antigens and more preferably four peptide antigens.

In an embodiment, the immunoreactivity is determined before preparation of a peptide vaccine to be used in the first administration protocol. In another embodiment, the immunoreactivity is further determined after the first administration protocol. Based on the result, a peptide antigen to be included in a peptide vaccine to be used in the next administration protocol may be selected.

The peptide vaccine includes, but not limited to, a short-chain peptide antigen, preferably an artificially synthesized short-chain peptide of 9 to 10 amino acids residues. In an embodiment, a peptide vaccine includes at least one peptide antigen selected from a peptide antigen group containing one or more SART2 peptides. In the specification, “SART2” refers to an enzyme involved in proteoglycan synthesis and known as dermatan sulfate epimerase 1 (DS-epi1). The one or more SART2 peptides may be, but not limited to, a peptide antigen consisting of the amino acid sequence at positions 93-101 of SART2 (DYSARWNEI (SEQ ID No. 1)) (hereinafter referred to also as “SART2-93”) and/or a peptide antigen consisting of the amino acid sequence at positions 161-169 of SART2 (AYDFLYNYL (SEQ ID No. 9)) (hereinafter referred to as “SART2-161”). In an embodiment, the one or more SART2 peptides comprise either one or both of SART2-93 and SART2-161. In another embodiment, the one or more SART2 peptides consist of either one or both of SART2-93 and SART2-161.

In an embodiment, a peptide vaccine includes at least one, preferably at least two, at least three, at least four or at most four peptide antigens selected from a peptide antigen group containing or consisting of peptide antigens shown in the following Table 1.

TABLE 1

| | Tumor- | | | |
|---|---|---|---|---|
| | associated | Amino | Amino | SEQ. |
| Peptide | antigen | acid | acid | ID. |
| antigen | protein | position | sequence | No. |
| SART2-93 | SART2 | 93-101 | DYSARWNEI | 1 |
| SART3-109 | SART3 | 109-118 | VYDYNCHVDL | 2 |
| Lck-208 | p56 lck | 208-216 | HYTNASDGL | 3 |
| PAP-213 | PAP | 213-221 | LYCESVHNF | 4 |
| PSA-248 | PSA | 248-257 | HYRKWIKDTI | 5 |
| EGF-R-800 | EGF-R | 800-809 | DYVREHKDNI | 6 |
| MRP3-503 | MRP3 | 503-511 | LYAWEPSFL | 7 |
| MRP3-1293 | MRP3 | 1293-1302 | NYSVRYRPGL | 8 |
| SART2-161 | SART2 | 161-169 | AYDFLYNYL | 9 |
| Lck-486 | p56 lck | 486-494 | TFDYLRSVL | 10 |
| Lck-488 | p56 lck | 488-497 | DYLRSVLEDF | 11 |
| PSMA-624 | PSMA | 624-632 | TYSVSFDSL | 12 |
| EZH2-735 | EZH2 | 735-743 | KYVGIEREM | 13 |
| PTHrP-102 | PTHrP | 102-111 | RYLTQETNKV | 14 |

In an embodiment, a peptide vaccine includes at least one, preferably at least two, at least three, at least four or at most four peptide antigens selected from the peptide antigen group containing or consisting of SART2-93, SART3-109, PAP-213, PSA-248, EGF-R-800, MRP3-503, MRP3-1293, SART2-161, Lck-486, Lck-488, PSMA-624 and PTHrP-102, among the peptide antigens shown in Table 1.

A peptide vaccine is prepared in accordance with a conventional method. The peptide vaccine may be prepared, for example, by mixing a peptide antigen(s) in the form of powder or liquid with a pharmaceutically acceptable carrier. In an embodiment, a peptide vaccine may further include an adjuvant for enhancing a specific immune response induced by the peptide antigen.

In the specification, the “adjuvant” refers to a substance or an auxiliary agent for enhancing a specific immune response to a peptide antigen included in a peptide vaccine and it is added to/mixed with the vaccine or simultaneously administered with the vaccine. Adjuvants are generally divided roughly into two types, i.e., an innate immune receptor activating type and a delivery system type. Examples of the innate immune receptor activating adjuvant include substances derived from components of microbes such as bacteria, virus and fungus, and derivatives thereof. Examples of the delivery system type adjuvant include a mineral salt such as aluminum salt, a water-oil-base emulsion and a liposome. The peptide vaccine may include, either one or both of the innate immune receptor activating adjuvant and the delivery system type adjuvant.

In an embodiment, a peptide vaccine is administered to a subject by subcutaneous injection. An injectable peptide vaccine may be prepared in accordance with a conventional method. The peptide vaccine may be prepared, for example, by dissolving a peptide antigen in the form of powder or liquid in a pharmaceutically acceptable injection solvent.

In an embodiment, a peptide vaccine may be administered in series which consists of 6 or 8 doses per course. When a protocol of 6 doses per course is employed, weekly administration is employed in the first course, every two weeks administration is employed in the second course, and administration at intervals of 2 weeks or more may be employed on and after the third course. Alternatively, when a protocol of 8 doses per course is employed, 1st to 4th doses of the first course may be administered weekly, the latter 4 doses are administered every two weeks; administration is carried out at intervals of 4 weeks in the second course; administration is carried out at intervals of 4 weeks or more on and after the third course. When a peptide vaccine includes, for example, 4 peptide antigens, the peptide vaccine may consist of 4 independent peptide vaccine compositions of the peptide antigens, and the peptide vaccine compositions may be administered independently by subcutaneous administration at separate sites (i.e., 4 sites).

In the specification, the “study product” refers to a tailor-made peptide vaccine according to the present invention to be administered in clinical trials (hereinafter also referred to as an “active drug”), or a placebo that does not comprise peptide antigens but comprises a corresponding adjuvant.

In the specification, “prognostic factor” refers to a factor that determines prognosis of a subject (a human suffering from a brain tumor) to a tailor-made peptide vaccine, in other words, a factor that predicts the effect of a tailor-made peptide vaccine.

In the present invention, the prognostic factor is selected from the group consisting of GM-CSF, at least one SART2, MCP-1, VEGF, IL-6, IL-7, IL-10, IL-17, IL-1RA, CCL4 and haptoglobin. In an embodiment, at least two, at least three, at least four, at least five, or five or more prognostic factors selected from the above group may be used.

In the case where at least two prognostic factors are selected, when one of the prognostic factors is selected from IL-17, CCL2, VEGF and IL-6, the rest of the prognostic factors may be selected from prognostic factors other than the prognostic factors (i.e., a group consisting of GM-CSF, at least one SART2, IL-7, haptoglobin (Hp), CCL4, IL-1RA and IL-10). In another example, when one of the prognostic factors is selected from CCL2, VEGF and IL-6, the rest of the prognostic factors may be selected from, prognostic factors other than the prognostic factors (i.e., a group consisting of GM-CSF, at least one SART2, IL-7, IL-17, haptoglobin (Hp), CCL4, IL-1RA and IL-10).

Among the above prognostic factors, GM-CSF, an immunoreactivity to at least one SART2, and MCP-1 are preferable because these prognostic factors can predict the therapeutic effect of a tailor-made peptide vaccine. In an embodiment, the prognostic factor is at least one selected from the group consisting of GM-CSF, an immunoreactivity to at least one SART2, and MCP-1. In an embodiment, the prognostic factor is GM-CSF, an immunoreactivity to at least one SART2 or MCP-1. In another embodiment, the prognostic factor is a combination of GM-CSF and an immunoreactivity to at least one SART2, a combination of an immunoreactivity to at least one SART2 and MCP-1 or a combination of GM-CSF and MCP-1. In another embodiment, the prognostic factor is a combination of GM-CSF, an immunoreactivity to at least one SART2, and MCP-1.

In the specification, the “granulocyte-macrophage colony-stimulating factor (GM-CSF)” refers to a type of colony stimulating factors. GM-CSF is known to act on granulocytes and precursor cells of macrophages (CFU-GM) to facilitate proliferation and differentiation of the cells; and acts on eosinophils and precursor cells of megakaryocytes to facilitate colony formation.

In an embodiment, the GM-CSF level of a subject's blood sample (for example, whole blood, plasma or serum) can be quantitatively measured in accordance with a conventional method. The GM-CSF level is, but not limited to, a GM-CSF concentration or GM-CSF content. In an embodiment, the GM-CSF level is the GM-CSF concentration.

GM-CSF can be measured in any timing and any number of times depending on the purpose of the measurement. GM-CSF may be measured, before, during or after preparation of a peptide vaccine or before the administration of the peptide vaccine. In an embodiment, GM-CSF is measured before preparation of a peptide vaccine. In another embodiment, GM-CSF may be measured during administration protocol and further before initiation of a next administration protocol.

In the specification, “MCP-1” is an abbreviation of Monocyte Chemoattractant Protein-1 and refers to a monocyte chemotactic factor produced by cells such as monocytes, vascular endothelial cells, and glioma cell lines. MCP-1 is also called as CCL-2. In an embodiment, the MCP-1 level of a subject's blood sample (for example, whole blood, plasma or serum) can be quantitatively measured in accordance with a conventional method. The MCP-1 level is, but not limited to, an MCP-1 concentration or MCP-1 content. In an example, the MCP-1 level is MCP-1 concentration.

MCP-1 may be measured in any timing and any number of times depending on the purpose of the measurement. MCP-1 may be measured before, during or after preparation of a peptide vaccine or before administration of the peptide vaccine. In an embodiment, MCP-1 is measured before preparation of a peptide vaccine. In another embodiment, MCP-1 may be measured during administration protocol and further before initiation of a next administration protocol.

The immunoreactivity of a subject to SART2 peptide may be quantitatively measured by any method, for example, in accordance with a conventional method. In an embodiment, the immunoreactivity of a subject to SART2 peptide may be measured by immunoassay (for example, ELISA), flow cytometry or flowmetry using subject's blood sample (for example, whole blood, plasma or serum). The immunoreactivity of a subject to SART2 peptide may be measured by using a substrate (for example, microtiter plate or bead) onto which SART2 peptide is immobilized so as to generate a predetermined level of a signal (for example, 1000 FIU) in response to a predetermined positive specimen (blood sample whose reactivity is known). In this case, the immunoreactivity can be measured by allowing the substrate to be in contact with a blood sample from a subject, washing the substrate, allowing the substrate to be in contact with an anti-human antibody labeled with a marker substance (for example, fluorescence substance) (labeled secondary antibody), and washing the substrate, and detecting a signal (for example, fluorescence) derived from the marker substance (for example, fluorescence substance). In this case, the immunoreactivity may be evaluated as being “no reactivity” or “not detected (ND)” when the level of a signal derived from the substrate (for example, fluorescence intensity FIU) is less than 10 FIU, and as being positive when the level is 10 FIU or more.

The immunoreactivity to SART2 peptide may be measured in any timing and any number of times depending on the purpose of the measurement. The immunoreactivity to SART2 peptide may be measured before, during or after preparation of a peptide vaccine or before administration of a peptide vaccine. In an embodiment, an immunoreactivity to SART2 peptide is measured before preparation of a peptide vaccine. In another embodiment, an immunoreactivity to SART2 peptide may be further measured during an administration protocol or before initiation of the following administration protocol.

In the specification, “vascular endothelial growth factor (VEGF)” refers to a type of vasculogenic factors. VEGF is known to be involved in vasculogenesis and neoangiogenesis.

In the specification, “interleukin 6 (IL-6)” refers to a glycoprotein consisting of 184 amino acid residues produced by various types of cells, such as T cells, B cells, macrophages and fibroblasts. IL-6 is known to be involved in events such as T/B cell proliferation/differentiation, acute-phase protein production, and heat generation.

In the specification, “interleukin 7 (IL-7)” refers to a glycoprotein consisting of 152 amino acid residues and produced by stromal cells and dendritic cells. IL-7 is known to be involved in events such as proliferation of T/B precursor cells and maintenance of homeostasis of mature T cells.

In the specification, “interleukin 10 (IL-10)” refers to a homodimer protein consisting of 160 amino acid residues and produced by T cells, macrophages and dendritic cells. IL-10 is known to be involved in events such as inhibition of macrophage function and activation of B cells.

In the specification, “interleukin 17 (IL-17)” refers to a protein consisting of 132 amino acid residues and produced by CD4 memory T cells and Th17 cells. IL-17 is known to be involved in events such as production of inflammatory cytokines from macrophages, epithelial cells, endothelial cells and fibroblasts.

In the specification, “interleukin 1RA (IL-1RA)” refers to a protein consisting of 152 amino acid residues produced by monocytes, macrophages, neutrophils and hepatocytes. IL-1RA is known to be involved in inhibition of IL-1 function by receptor competition.

In the specification, “CCL4” refers to a cytokine produced by cells such as macrophages, monocytes, and dendritic cells. CCL4 is known to be involved in induction of monocytes, natural killer cells and memory T cells.

In the specification, “haptoglobin (Hp)” refers to a protein produced by mature granulocytes (particularly eosinophil) such as hepatocytes and lymph nodes. Haptoglobin is known to have a specific affinity for hemoglobin and inhibit urinary excretion of hemoglobin.

In an embodiment, the levels of IL-6, IL-7, IL-10, IL-17, IL-1RA, CCL-4 and haptoglobin in a subject's blood sample (for example, whole blood, plasma or serum) each may be quantitatively measured in accordance with a conventional method. The levels of these factors refer to, but not limited to, concentrations or contents. In an embodiment, the levels of these factors refer to concentrations.

The level of IL-6, IL-7, IL-10, IL-17, IL-1RA, CCL-4 or haptoglobin may be measured in any timing and any number of times depending on the purpose of the measurement. Measurement of IL-6, IL-7, IL-10, IL-17, IL-1RA, CCL-4 or haptoglobin may be carried out before, during or after preparation of a peptide vaccine or before administration of the peptide vaccine. In an embodiment, measurement of IL-6, IL-7, IL-10, IL-17, IL-1RA, CCL-4 or haptoglobin is carried out before administration of a peptide vaccine. In another embodiment, measurement of IL-6, IL-7, IL-10, IL-17, IL-1RA, CCL-4 or haptoglobin may be further carried out during administration protocol and further carried out before initiation of a next administration protocol.

[Evaluation Step]

In an embodiment, an evaluation step comprises comparing GM-CSF level (for example, GM-CSF concentration) in a subject's blood sample (for example, whole blood, plasma or serum) with a predetermined GM-CSF threshold (hereinafter referred to as “GM-CSF threshold”). In the evaluation step, an evaluation is given as “having no risk” when the GM-CSF level is less than the GM-CSF threshold; whereas, an evaluation is given as “having a risk” when the GM-CSF level is the GM-CSF threshold or more (hereinafter referred to also as “evaluation A”).

The GM-CSF threshold is, but not limited to, a value of GM-CSF level by which the score of a subject group (a plurality of human subjects suffering from a brain tumor) treated with an immunotherapy by administration of a peptide vaccine (overall survival of active-drug group) and the score of a subject group untreated or given a placebo (overall survival of placebo group) are divided, respectively, such that the divided active-drug group's overall survival below the threshold is more likely distinguished, or preferably statistically significantly distinguished, from the divided placebo group's overall survival below the threshold. The statistically significant difference may be analyzed by a test method known in the art. A test method may be, but not limited to, a log-rank test. In an embodiment, the GM-CSF threshold is 0.9 pg/mL.

In an embodiment, the GM-CSF threshold is a value of GM-CSF level by which the overall survival of the active-drug group is statistically significantly divided into a group that exhibits a life extension effect (hereinafter referred to also as “effective group”) and a group that does not exhibit a life extension effect (hereinafter referred to also as “ineffective group”). The statistical significance may be analyzed by a test method known in the art. A test method may be, but not limited to, a long-rank test. In an embodiment, the GM-CSF threshold is a value by which a part of the subjects corresponding to a certain percentage (for example, 20%, 15%, 10% or 8%) from the top of GM-CSF level in the subject group can be distinguished. The GM-CSF threshold is, for example, 5 pg/mL, 3 pg/mL or 2 pg/mL.

In the present invention, the number of GM-CSF threshold values is not limited to one. For example, when a group of the active-drug group on which an immunotherapy effectively works can be statistically significantly further divided into a group that shows a large life extension effect and a group that shows a small life extension effect based on a certain value, the value may be defined as a secondary threshold. In an embodiment, the GM-CSF threshold may include a first threshold of GM-CSF (hereinafter referred to also as “GM-CSF threshold (1)”) and a second threshold of GM-CSF that is smaller than GM-CSF threshold (1) (hereinafter referred to also as “GM-CSF threshold (2)”). In this case, the evaluation A is given as “having a risk” when the GM-CSF concentration of a subject is not less than the GM-CSF threshold (1); as “life extension effect is promising” when the GM-CSF concentration of a subject is less than the GM-CSF threshold (1) and not less than the GM-CSF threshold (2); and as “satisfactory life extension effect is promising” when the GM-CSF concentration of a subject is less than the GM-CSF threshold (2).

In an embodiment, an evaluation step comprises comparing a value showing an immunoreactivity of a subject to at least one SART2 peptide with a predetermined threshold regarding SART2 (hereinafter referred to as “SART2 threshold”). In the comparison, an evaluation is given as “having no risk” when the value showing the immunoreactivity to the at least one SART2 peptide is less than the SART2 threshold; whereas, an evaluation is given as “having a risk” when the value showing the immunoreactivity to any one of the at least one SART2 peptide is not less than the SART2 threshold (hereinafter referred to also as “evaluation B”).

The SART2 threshold is, but not limited to, a value showing immunoreactivity to SART2 by which the overall survival of the active-drug group is statistically significantly divided into an effective group and an ineffective group. The statistical significance may be analyzed by a test method known in the art. A test method may be, but not limited to, a log-rank test.

In the present invention, the number of SART2 threshold values is not limited to one. For example, when a subgroup of the active-drug group on which an immunotherapy effectively works can be statistically significantly further divided into a group that shows a large life extension effect and a group that shows a small life extension effect based on a certain value, the value may be defined as a secondary threshold. In an embodiment, the SART2 threshold may include a first threshold of SART2 (hereinafter referred to also as “SART2 threshold (1)”) and a second threshold of SART2 that is smaller than a SART2 threshold (1) (hereinafter referred to also as “SART2 threshold (2)”). In this case, an evaluation may be given as “having a risk” when the SART2 level of a subject is not less than the SART2 threshold (1); as “life extension effect is promising” when the SART2 level of a subject is less than the SART2 threshold (1) and not less than the SART2 threshold (2); and as “satisfactory life extension effect is promising” when the SART2 level of a subject is less than the SART2 threshold (2).

In another case, the SART2 threshold may represent a value showing the 5th immunoreactivity, the 4th immunoreactivity, the 3rd immunoreactivity, the 2nd immunoreactivity or the 1st immunoreactivity among the immunoreactivities of a subject to each of the peptide antigens constituting a peptide antigen group including SART2 peptide (for example, a peptide antigen group consisting of 14 peptide antigens shown in Table 1). In an embodiment, when a peptide vaccine is prepared so as to include 4 peptide antigens having a 1st to 4th highest immunoreactivity from peptide antigens that constitute a peptide antigen group, the 4th immunoreactivity value is employed as the SART2 threshold. In this case, when the immunoreactivity of a subject to SART2 peptide is not less than the SART2 threshold (i.e., the value showing 4th immunoreactivity), an evaluation is given as “having a risk” while the peptide vaccine includes the SART2 peptide. In other words, an evaluation is given as “having a risk” when a peptide vaccine includes SART2 peptide; and an evaluation is given as “having no risk” when a peptide vaccine does not include SART2 peptide.

In an embodiment, at least one SART2 peptide may be either one or both of SART2-93 peptide (SEQ ID No. 1) and SART2-161 peptide (SEQ ID No. 9). In this case, an evaluation is given as “having a risk” in the evaluation B when the peptide vaccine includes any one of the SART2 peptides, more specifically, at least one of SART2-93 peptide (SEQ ID No. 1) and SART2-161 peptide (SEQ ID No. 9). In another case where the peptide vaccine does not include the SART2 peptide, in other words, the peptide vaccine includes neither SART2-93 peptide (SEQ ID No. 1) nor SART2-161 peptide (SEQ ID No. 9), an evaluation is given as “having no risk” in the evaluation B.

In an embodiment, an evaluation step comprises comparing MCP-1 level (for example, MCP-1 concentration) in a subject's blood sample (for example, whole blood, plasma or serum) with a predetermined MCP-1 threshold being first MCP-1 threshold (hereinafter referred to as “MCP-1 threshold (1)”). In the comparison, for example, an evaluation is given as “having a risk” when the MPC-1 level is less than the MCP-1 threshold (1). In another embodiment, an evaluation step comprises comparing the MCP-1 level with a predetermined MCP-1 threshold including MCP-1 (1) and a second threshold that is greater than the MCP-1 threshold (1) (hereinafter referred to also as “MCP-1 threshold (2)”); giving an evaluation as “having no risk” when the MPC-1 level is not less than the MCP-1 threshold (1) and less than the MCP-1 threshold (2), and giving an evaluation as “having a risk” when the MCP-1 level is less than the MCP-1 threshold (1) or not less than the MCP-1 threshold (2) (hereinafter referred to also as “evaluation C”).

The MCP-1 threshold (1) and MCP-1 threshold (2) are, but not limited to, a value showing an MCP-1 level by which the overall survival of the active-drug group is statistically significantly divided into an effective group and an ineffective group. The statistical significance may be analyzed by a test method known in the art. A test method may be, but limited to, a log-rank test.

In an embodiment, the MCP-1 threshold (1) is a value by which a part of the subjects corresponding to a certain percentage (for example, 15%, 10% or 8%) from the lowest MCP-1 level in the subject group can be distinguished. In another embodiment, the MCP-1 threshold (1) is 75 pg/mL, 100 pg/mL or 125 pg/mL.

In an embodiment, the MCP-1 threshold (2) is a value by which a part of the subjects corresponding to a certain percentage (for example, 15%, 10% or 8%) from the highest MCP-1 level in the subject group can be distinguished. In another embodiment, the MCP-1 threshold (2) is 650 pg/mL, 700 pg/mL or 750 pg/mL.

The MCP-1 threshold (1) and MCP-1 threshold (2) may be, but not limited to, any combination of the MCP-1 levels described above. In an embodiment, the MCP-1 threshold (1) is a value showing the level of 100 pg/mL; whereas, the MCP-1 threshold (2) is a value showing the level of 700 pg/mL. In another embodiment, the MCP-1 threshold (1) is a value showing the level that distinguishes the part of the subjects corresponding to 10% from the top in the subject group; whereas, the MCP-1 threshold (2) is a value showing the level that distinguishes the part of the subjects corresponding to 10% from the lowest in the subject group.

The number of MCP-1 threshold values may be, but not limited to, a single one. In an embodiment, when the MCP-1 level of a subject is less than a median value of the MCP-1 level of an active-drug group, the MCP-1 threshold may be the value showing the level of the MCP-1 threshold (1) described above. In this case, an evaluation is given as “having a risk” when the MCP-1 level of a subject is less than the MCP-1 threshold; and as “having no risk” when the MCP-1 level is not less than the MCP-1 threshold. In another embodiment, when the MCP-1 level of a subject is not less than a median value of the MCP-1 level of an active-drug group, the MCP-1 threshold may be the value showing the level of the MCP-1 threshold (2) described above. In this case, an evaluation is given as “having no risk” when the MCP-1 level of a subject is less than an MCP-1 threshold; and as “having a risk” when the MCP-1 level is not less than the MCP-1 threshold.

In the present invention, the number of MCP-1 threshold values is not limited to one or two. For example, when a group of the active-drug group on which an immunotherapy effectively works can be statistically significantly further divided into a group that shows a large life extension effect and a group that shows a small life extension effect based on a certain value, the value may be defined as a third threshold. In an embodiment, the MCP-1 threshold may include a third threshold (hereinafter referred to also as “MCP-1 threshold (3)”) between the MCP-1 threshold (1) and the MCP-1 threshold (2). In this case, when the MCP-1 level of a subject is less than the MCP-1 threshold (1) or not less than the MCP-1 threshold (2), an evaluation may be given as “having a risk”. In this case, a group for which an evaluation is given as “having no risk” (i.e., a group showing an MCP-1 level that is not less than the MCP-1 threshold (1) and less than the MCP-1 threshold (2)) may be further subjected to evaluation carried out on the basis of the MCP-1 threshold (3). In this case, an evaluation is given as “life extension effect is promising” when the MCP-1 level is less than the MCP-1 threshold (3); and as “satisfactory life extension effect is promising” when the MCP-1 level is not less than the MCP-1 threshold (3).

When the MCP-1 threshold includes the MCP-1 threshold (1) and the MCP-1 threshold (2), and the subjects of the active-drug group and a placebo group are divided, respectively, into a group showing a MCP-1 level that is not less than the threshold (1) and less than the threshold (2) (also referred to as “group A”) and a group showing a MCP-1 level that is less than a threshold (1) or not less than the threshold (2) (also referred to as “group B”), the MCP-1 thresholds (1) and (2) may be the values of MCP-1 levels by which the overall survival of group A of the active-drug group and the overall survival of group A of a placebo group are more likely distinguished or preferably statistically significantly distinguished based on the thresholds. The statistically significant difference may be analyzed by a test method known in the art. A test method may be, but not limited to, a log-rank test. In this embodiment, the MCP-1 threshold (1) and the MCP-1 threshold (2) may be the values showing the levels described above.

When the prognostic factor is VEGF, IL-7, IL-17, IL-6 or haptoglobin, the number of threshold values of the prognostic factor may be, but not limited to, 1, 2 or 3, similar to the case of MCP-1. Evaluation using the prognostic factor comprises comparing the level (for example, concentration) of the prognostic factor in a subject's blood sample (for example, whole blood, plasma or serum) with a predetermined threshold including a first threshold (referred to as “threshold (1)”) and a second threshold greater than the threshold (1) (hereinafter referred to also as “threshold (2)”) of the prognostic factor. In the comparison, an evaluation is given as “having no risk” when the level of the prognostic factor of a subject is not less than the threshold (1) and less than the threshold (2); and as “having a risk” when the level of the prognostic factor of a subject is less than the threshold (1) or not less than the threshold (2).

The threshold (1) and threshold (2) of the prognostic factor is, but not limited to, a value showing the level of the prognostic factor by which the overall survival of an active-drug group is statistically significantly divided into an effective group and an ineffective group. The statistical significance may be analyzed by a test method known in the art. A test method may be, but not limited to, a log-rank test.

The threshold (1) of the prognostic factor is, but not limited to, a value by which a part of subjects corresponding to a certain percentage (for example, 15%, 10%, 8%, 5% or 3%) from the lowest prognostic factor level in the subject group can be distinguished. Threshold (2) of each prognostic factor is, but not limited to, a value by which a part of subjects corresponding to a certain percentage (for example, 15%, 10%, 8%, 5% or 3%) from the lowest prognostic factor level in the subject group can be distinguished.

A VEGF threshold (1) is, but not limited to, 2 pg/mL, 3 pg/mL or 5 pg/mL. A VEGF threshold (2) is, but not limited to, 10 pg/mL, 15 pg/mL or 20 pg/mL.

A haptoglobin threshold (1) is, but not limited to, 160 μg/mL, 180 μg/mL or 200 μg/mL. A haptoglobin threshold (2) is, but not limited to, 1000 μg/mL, 1200 μg/mL or 1400 μg/m.

An IL-6 threshold (1) is, but not limited to, 1 pg/mL, 1.5 pg/mL or 2 pg/mL. An IL-6 threshold (2) is, but not limited to, 7 pg/mL, 9 pg/mL or 11 pg/mL.

When the prognostic factor is CCL4, IL-1RA or IL-10, the number of threshold values of the prognostic factor may be, but not limited to, one or two, similar to the case of GM-CSF. For example, evaluation using the prognostic factor comprises comparing the level (for example, concentration) of the prognostic factor in a subject's blood sample (for example, whole blood, plasma or serum) with a predetermined threshold of the prognostic factor (simply referred to as “threshold”). In the comparison, an evaluation is given as “having no risk” or “having a risk” when the level of the prognostic factor of a subject is less than the threshold; and as “having a risk” or “having no risk” when the level of the prognostic factor is not less than the threshold.

The threshold of the prognostic factor is, but not limited to, a value showing the level of the prognostic factor by which the overall survival of an active-drug group is statistically significantly divided into an effective group and ineffective group. The statistical significance may be analyzed by a test method known in the art. A test method may be, but not limited to, a log-rank test. In an embodiment, the threshold of the prognostic factor may be a median value of the level of the prognostic factor in an active-drug group. In another embodiment, the threshold of the prognostic factor is a value by which a part of subjects corresponding to a certain percentage (for example, 20%, 15%, 10% or 8%) from the highest prognostic factor level in the active-drug group can be distinguished.

The CCL4 threshold is, but not limited to, a median value of the CCL4 level in an active-drug group. In an embodiment, the CCL4 threshold is a value showing CCL4 level by which the part of the subjects corresponding to 10% from the highest in the active-drug group can be distinguished. In an embodiment, the IL-1RA threshold is a value showing IL-1RA level by which the part of the subjects corresponding to 10% from the highest in the active-drug group can be distinguished. In an embodiment, the IL-10 threshold is a value showing IL-10 level by which the part of the subjects corresponding to 10% from the highest in the active-drug group can be distinguished.

The threshold of the prognostic factor is determined, for example, in an active-drug group of 20 subjects or more and/or a placebo group of 20 subjects or more. In an embodiment, the threshold of the prognostic factor is determined in an active-drug group of 30 subjects or more, 50 subjects or more and 100 subjects or more and/or a placebo group of 30 subjects or more, 50 subjects or more and 100 subjects or more.

In an evaluation step, expression of evaluation is not limited to “having no risk” and “having a risk”. For example, the phrase “having no risk” may be rephrased by “high possibility of producing an effect” or “therapeutic effect is promising”. The phrase “having a risk” may be rephrased by “low possibility of producing an effect” and “therapeutic effect is not promising”. The therapeutic effect may be represented, for example, by the survival time extended in compared with a median survival time of a placebo group.

In the present invention, the thresholds of prognostic factors may be categorized into sub-groups in accordance with the features of subjects. More specifically, the threshold may be separately set depending on the gender, age or race. In this case, a threshold is obtained from a group of thresholds predetermined depending on the gender, age and race of the subjects.

The evaluation step may comprise evaluation carried out based on at least one prognostic factor. In an embodiment, the evaluation step comprises evaluations based on at least two, at least three, at least four prognostic factors. In another embodiment, the evaluation step comprises evaluations based on one, two, three and four prognostic factors. In another embodiment, the evaluation step comprises evaluation based on at least one prognostic factor selected from the group consisting of GM-CSF, at least one SART2, and MCP-1. In an embodiment, the evaluation step comprises evaluations based on at least two prognostic factors selected from the group consisting of GM-CSF, at least one SART2, and MCP-1, at least one SART2, and MCP-1. When the evaluation step comprises, evaluations based on 2 or more prognostic factors, the evaluations may be carried out in any order.

The evaluation step may comprise the evaluation A and further comprise either one or both of the evaluation B and the evaluation C. More specifically, the evaluation step comprises the evaluation A and the evaluation B; the evaluation A and the evaluation C; or the evaluation A, the evaluation B and the evaluation C. When the evaluation step comprises two or more evaluations, the evaluations may be carried out in any order. For example, when the evaluation step comprises the evaluation A and the evaluation B, the evaluation A may be followed by the evaluation B or the evaluation B may be followed by the evaluation A. Alternatively, the evaluation A and the evaluation B may be simultaneously carried out.

[Determination Step]

In the determination step, whether a subject is eligible or ineligible for application of a tailor-made peptide vaccine is determined based on the evaluation in the evaluation step. In the determination step, for example, the subject is determined as an “eligible person” when an evaluation is given as “having no risk” in the evaluation step; and as an “ineligible person” when an evaluation is given as “having a risk”. In an embodiment, when the evaluation step comprises at least two evaluations, the subject is determined as an “eligible person” in the determination step when all of the at least two evaluations are given as “having no risk”; and the subject is determined as an “ineligible person” when all of the at least two evaluations are given as “having a risk”, or either one of the at least two evaluations is given as “having a risk”. In an embodiment, in the case where the evaluation step comprises the evaluation A, the evaluation B and the evaluation C, a subject is determined as an eligible person for the tailor-made peptide vaccine in the determination step when any one of the evaluation A, the evaluation B and the evaluation C is given as “having no risk”.

In an embodiment, in the case where the evaluation step comprises the evaluation A and the evaluation B, when either one or both of the evaluation A and the evaluation B is given as “having no risk”, the subject is determined as an eligible person for tailor made peptide vaccine in the determination step. In another embodiment, in the case where the evaluation step comprises the evaluation A and the evaluation C, when either one or both of the evaluation A and the evaluation C are given as “having no risk”, the subject is determined as an eligible person for tailor made peptide vaccine in the determination step. In another embodiment, in the case where the evaluation step comprises the evaluation A, the evaluation B and the evaluation C, when the evaluation A is given as “having no risk” and evaluation of either one or both of the evaluation B and C is given as “having no risk”, the subject is determined as an eligible person for tailor-made peptide vaccine in the determination step.

In an embodiment, in the case where the evaluation step comprises the evaluation A and the evaluation B, when both of the evaluation A and the evaluation B are given as “having a risk”, the subject is determined as an ineligible person for tailor made peptide vaccine in the determination step. In another embodiment, in the case where the evaluation step comprises the evaluation A and the evaluation C, when both of the evaluation A and the evaluation C are given as “having a risk”, the subject is determined as an ineligible person for tailor made peptide vaccine in the determination step. In another embodiment, in the case where the evaluation step comprises the evaluation A, the evaluation B and the evaluation C, the subject may be determined as an ineligible person for tailor made peptide vaccine in the determination step when all of the evaluations A to C are given as “having a risk”. In this embodiment, the subject may be determined as an ineligible person for tailor made peptide vaccine in the determination step when two of the evaluations A to C including the evaluation A are given as “having a risk”.

Expressions of determinations in the determination step are not limited to “eligible person” or “ineligible person” and may be any phrases. The expressions of determinations may also be appropriately determined depending on the combination of evaluations. For example, in the case where the evaluation step comprises evaluations based on 3 prognostic factors, when two of the evaluations are given as “having a risk” and the remaining evaluation is given as “having no risk”, the expression of determination may be “the tailor-made peptide vaccine is not recommended”. When all of the 3 evaluations are given as “having a risk”, the expression of determination may be “ineligible person”. For example, in the case where the evaluation step comprises the evaluations A to C, when two of the evaluations including the evaluation A (i.e., the evaluation A and the evaluation B or C) are given as “having a risk” and the remaining evaluation is given as “having no risk”, the expression of determination may be “not recommended”. When all of the evaluations A to C are given as “having a risk”, the expression of determination may be “ineligible person”.

In the determination step according to the present invention, when a subject is determined as an “eligible person”, it is recommended that the subject should receive an immunotherapy using the tailor-made peptide vaccine, and a brain tumor treatment by administering the peptide vaccine may be applied to the subject. Accordingly, another aspect of the present invention is to provide a method for treating a brain tumor by administering a peptide vaccine to a subject determined as an eligible person for the peptide vaccine by the above determination method.

In an aspect, the present invention provides a method for treating a subject suffering from a brain tumor by administering a tailor-made peptide vaccine including at least one peptide antigen, comprising the steps of evaluating a risk of a subject with regard to a peptide vaccine; determining whether the subject is an eligible person for the peptide vaccine based on the evaluation; and administering the peptide vaccine to the subject based on the determination, as described above. In an embodiment, the present invention provides a method for treating a subject suffering from a brain tumor by administering a tailor-made peptide vaccine including at least one peptide antigen, wherein the subject is a person who is determined as an eligible person for the peptide vaccine based on an evaluation on a risk of the subject with regard to the peptide vaccine; the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin. The features of the elements such as peptide antigens, peptide vaccines, evaluation steps, determination steps, brain tumors, subjects, administration methods and thresholds as described herein are also applied to this aspect of the invention.

In an aspect, the present invention provides a kit for determining whether a subject suffering from a brain tumor is an eligible person for a tailor-made peptide vaccine including at least one peptide antigen, containing a reagent for measuring at least one prognostic factor selected from the group consisting of GM-CSF, at least one SART2, MCP-1 (CCL2), VEGF, IL-7, IL-17, IL-6, haptoglobin (Hp), CCL4, IL-1RA, and IL-10. The kit be the one that can quantitatively measure a prognostic factor. In an embodiment, the kit is for measurement by ELISA, flow cytometry or flowmetry. The kit contains reagent(s) for quantitatively measuring at least one, at least two, at least three or at least four prognostic factors. The kit may be appropriately manufactured in accordance with a conventional method.

The reagent includes, but not limited to, antibodies specifically binding to individual prognostic factors. In an embodiment, the reagent includes antibodies each specifically binding to one of at least one, at least two or at least three prognostic factors selected from GM-CSF, at least one SART2 and MCP-1 (CCL2). In an embodiment, the reagent includes at least one bead onto which at least one antibody selected from the above antibodies is immobilized. The bead onto which the antibody is immobilized may be obtained by immobilizing the antibody against a certain antigen onto a bead in accordance with a conventional method. Examples of the bead include, but not limited to, fluorescent beads, which may be manufactured in accordance with a conventional method or commercially available. The antibody against a certain antigen may be obtained in accordance with a conventional method. The reagent for quantitatively measuring the prognostic factor may further include, but not limited to, a buffer, a color reagent and a washing agent.

In an embodiment, the present invention provides a kit for use in the treatment of a brain tumor for preparing a tailor-made peptide vaccine including at least one, at least two, at least three or at most four peptide antigens selected from the group consisting of 14 peptide antigens shown in Table 1, or from the group consisting of SART2-93, SART3-109, PAP-213, PSA-248, EGF-R-800, MRP3-503, MRP3-1293, SART2-161, Lck-486, Lck-488, PSMA-624 and PTHrP-102, wherein the tailor-made peptide vaccine is administered to a subject determined as an eligible person by the determination method according to the present invention.

The kit contains 14 peptide antigens shown in Table 1 or 12 peptide antigens of SART2-93, SART3-109, PAP-213, PSA-248, EGF-R-800, MRP3-503, MRP3-1293, SART2-161, Lck-486, Lck-488, PSMA-624 and PTHrP-102, in the form of powder or liquid. The kit may further contain, for example, a pharmaceutically acceptable carrier and an adjuvant for enhancing specific immune response induced by a peptide antigen. The kit may be appropriately manufactured in accordance with a conventional method. The features of the elements as such as peptide vaccines and adjuvants as described herein are also applied to this aspect of the invention.

Embodiments of the present invention may be, for example, those described below, but not limited thereto:

[Item 1] A method for determining whether a subject suffering from a brain tumor is an eligible person for a tailor-made peptide vaccine composition including at least one peptide antigen, comprising the steps of: evaluating a risk of the subject with respect to the peptide vaccine composition; and determining whether the subject is an eligible person for the peptide vaccine composition based on the evaluation, wherein the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin.

[Item 2] The determination method according to Item 1, wherein the prognostic factor includes at least one selected from GM-CSF, the at least one SART2, and MCP-1.

[Item 3] The determination method according to Item 1 or 2, wherein the prognostic factor includes at least two selected from the group.

[Item 4] The determination method according to Item 1, wherein the evaluation step comprises evaluating a risk of the subject with respect to the peptide vaccine composition by comparing a level of granulocyte-macrophage colony-stimulating factor (GM-CSF) in a blood sample from the subject with a GM-CSF threshold (evaluation A); and further comprises either one or both of evaluating a risk of the subject with respect to the peptide vaccine composition by comparing an immunoreactivity of the subject to the at least one SART2 peptide with a SART2 threshold (evaluation B) and evaluating a risk of the subject with respect to the peptide vaccine composition by comparing a level of Monocyte Chemoattractant Protein-1 (MCP-1) in a blood sample from the subject with a MCP-1 threshold (evaluation C); wherein in the evaluation A, an evaluation is given as “having no risk” when the GM-CSF level is less than the GM-CSF threshold, and an evaluation is given as “having a risk” when the GM-CSF level is not less than the GM-CSF threshold, in the evaluation B, an evaluation is given as “having no risk” when the immunoreactivities of the subject to both of the at least one SART2 peptide are less than the SART2 threshold, and an evaluation is given as “having a risk” when the immunoreactivity of the subject to any one of the at least one SART2 peptide is not less than the SART2 threshold, and in the evaluation C, in the case where the MCP-1 threshold includes an MCP-1 threshold (1), an evaluation is given as “having a risk” when the MPC-1 level is less than the MCP-1 threshold (1); or in the case where the MCP-1 threshold includes an MCP-1 (1) and an MCP-1 threshold (2) that is greater than the value of the MCP-1 (1), an evaluation is given as “having no risk” when the MCP-1 level is not less than the MCP-1 threshold (1) and is less than the MCP-1 threshold (2); and an evaluation is given as “having a risk” when the MCP-1 level is less than the MCP-1 threshold (1) or not less than the MCP-1 threshold (2).

[Item 5] The determination method according to Item 4, wherein the subject is determined as an “eligible person” for the peptide vaccine composition in the determination step, when the evaluation step comprises the evaluation A and the evaluation B, and either one or both of the evaluation A and the evaluation B are given as “having no risk”; or when the evaluation step comprises the evaluation A and the evaluation C, and either one or both of the evaluation A and the evaluation C are given as “having no risk”.

[Item 6] The determination method according to Item 4, wherein the subject is determined as an “ineligible person” for the peptide vaccine composition in the determination step, when the evaluation step comprises the evaluation A and the evaluation B, and both of the evaluation A and the evaluation B are given as “having a risk”; when the evaluation step comprises the evaluation A and the evaluation C, and both of the evaluation A and the evaluation C are given as “having a risk”; or when the evaluation step comprises the evaluation A, the evaluation B, and the evaluation C, and the evaluation A, the evaluation B, and the evaluation C are all given as “having a risk”.

[Item 7] The determination method according to Item 5 or 6, wherein the at least one SART2 peptide includes either one or both of SART2-93 peptide (SEQ ID No. 1) and SART2-161 peptide (SEQ ID No. 9).

[Item 8] The determination method according to any one of Items 1 to 7, wherein the subject is HLA-A24 positive; the peptide vaccine composition includes at least two peptide antigens selected from the peptide antigen group containing SART2-93 peptide (SEQ ID No. 1), SART3-109 peptide (SEQ ID No. 2), Lck-208 peptide (SEQ ID No. 3), PAP-213 peptide (SEQ ID No. 4), PSA-248 peptide (SEQ ID No. 5), EGF-R-800 peptide (SEQ ID No. 6), MRP3-503 peptide (SEQ ID No. 7), MRP3-1293 peptide (SEQ ID No. 8), SART2-161 peptide (SEQ ID No. 9), Lck-486 peptide (SEQ ID No. 10), Lck-488 peptide (SEQ ID No. 11), PSMA-624 peptide (SEQ ID No. 12), EZH2-735 peptide (SEQ ID No. 13) and PTHrP-102 peptide (SEQ ID No. 14), and the at least two peptide antigens are selected in the descending order of immunoreactivities of the subject to the peptide antigens.

[Item 9] The determination method according to any one of Items 1 to 8, wherein the brain tumor is glial tumor or glioma.

[Item 10] The determination method according to Item 9, wherein the brain tumor is glioma, and the glioma is resistant to a temozolomide therapy.

[Item 11] The determination method according to any one of Items 1 to 10, wherein the peptide vaccine composition includes at most four peptide antigens.

[Item 12] The determination method according to any one of Items 1 to 11, further comprising the steps of: evaluating a risk with respect to administration of the tailor-made peptide vaccine composition based on a lymphocyte in a blood sample from the subject; and determining whether the administration of the peptide vaccine composition is canceled or not based on the evaluation, wherein the lymphocyte is at least one selected from the group consisting of a CD11b⁺CD14⁺HLA-DR^{low}-immunosuppressive monocyte, a CD3⁺CD4⁺CD45RA⁻T cell and a CD4⁺CD25⁺FoxP3⁺ cell (Treg).

[Item 13] A method for treating a subject suffering from a brain tumor by administering a tailor-made peptide vaccine composition including at least one peptide antigen, wherein the subject is a person who is determined as an eligible person for the peptide vaccine composition based on an evaluation on a risk of the subject with regard to the peptide vaccine composition; and the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin.

[Item 14] The treatment method according to Item 13, wherein the peptide vaccine composition includes at least two peptide antigens selected from the peptide antigen group containing SART2-93 peptide (SEQ ID No. 1), SART3-109 peptide (SEQ ID No. 2), Lck-208 peptide (SEQ ID No. 3), PAP-213 peptide (SEQ ID No. 4), PSA-248 peptide (SEQ ID No. 5), EGF-R-800 peptide (SEQ ID No. 6), MRP3-503 peptide (SEQ ID No. 7), MRP3-1293 peptide (SEQ ID No. 8), SART2-161 peptide (SEQ ID No. 9), Lck-486 peptide (SEQ ID No. 10), Lck-488 peptide (SEQ ID No. 11), PSMA-624 peptide (SEQ ID No. 12), EZH2-735 peptide (SEQ ID No. 13), and PTHrP-102 peptide (SEQ ID No. 14).

[Item 15] A kit for use in the determination method according to any one of Items 1 to 12, containing a reagent for measuring at least one prognostic factor selected from the group consisting of GM-CSF, at least one SART2, MCP-1, VEGF, IL-6, IL-7, IL-10, IL-17, IL-1RA, CCL4, and haptoglobin.

[Item 17] The determination method according to Item 3, wherein the evaluation is made based on at least two prognostic factors selected from the group consisting of GM-CSF, at least one SART2, MCP-1, VEGF, IL-6, IL-7, IL-10, IL-17, IL-1RA, CCL4 and haptoglobin, and when one of the prognostic factors is selected from a sub-group consisting of MCP-1, VEGF and IL-6, at least one of the rest of the prognostic factors is selected from the group consisting of GM-CSF, at least one SART2, IL-7, IL-10, IL-17, IL-1RA, CCL4 and haptoglobin.

[Item 18] The determination method according to any one of Items 1 to 3 and Items 9 to 12, wherein the evaluation step comprises any of the following evaluations depending on the prognostic factor selected:

evaluating a risk of the subject with regard to the peptide vaccine by comparing a VEGF level in a blood sample from the subject with a VEGF threshold, and in the evaluation, an evaluation is given as “having a risk” when the VEGF threshold includes a VEGF threshold (1) and the VEGF level is less than the VEGF threshold (1); or an evaluation is given as “having no risk” when the VEGF threshold includes a VEGF (1) and a VEGF threshold (2) that is greater than the VEGF (1) and the VEGF level is not less than the VEGF threshold (1) and less than the VEGF threshold (2), and as “having a risk” when the VEGF level is less than the VEGF threshold (1) or not less than the VEGF threshold (2),

evaluating a risk of the subject with regard to the peptide vaccine by comparing an IL-6 level in a blood sample from the subject with an IL-6 threshold, and in the evaluation, an evaluation is given as “having a risk” when the IL-6 threshold includes an IL-6 threshold (1) and the IL-6 level is less than the IL-6 threshold (1); or an evaluation is given as “having no risk” when the IL-6 threshold includes an IL-6 (1) and an IL-6 threshold (2) that is greater than the IL-6 (1), and the IL-6 level is not less than the IL-6 threshold (1) and less than the IL-6 threshold (2), and as “having a risk” when the IL-6 level is less than the IL-6 threshold (1) or not less than the IL-6 threshold (2),

evaluating a risk of the subject with regard to the peptide vaccine by comparing an IL-7 level in a blood sample from the subject with an IL-7 threshold, and in the evaluation, an evaluation is given as “having a risk” when the IL-7 threshold includes an IL-7 threshold (1) and the IL-7 level is less than the IL-7 threshold (1); or an evaluation is given as “having no risk” when the IL-7 threshold includes IL-7 (1) and an IL-7 threshold (2) that is greater than the IL-7 (1), and the IL-7 level is not less than the IL-7 threshold (1) and less than the IL-7 threshold (2), and as “having a risk” when the IL-7 level is less than the IL-7 threshold (1) or not less than the IL-7 threshold (2),

evaluating a risk of the subject with regard to the peptide vaccine by comparing an IL-10 level in a blood sample from the subject with an IL-10 threshold, and in the evaluation, an evaluation is given as “having no risk” when the IL-10 level is less than the IL-10 threshold, and a as “having a risk” when IL-10 level is not less than the IL-10 threshold,

evaluating a risk of the subject with regard to the peptide vaccine by comparing an IL-17 level in a blood sample from the subject with an IL-17 threshold, and in the evaluation, an evaluation is given as “having a risk” when the IL-17 threshold includes an IL-17 threshold (1) and the IL-17 level is less than the IL-7 threshold (1); or an evaluation is given as “having no risk” when the IL-17 threshold includes IL-17 (1) and an IL-17 threshold (2) that is greater than the IL-17 (1) and the IL-17 level is not less than the IL-17 threshold (1) and less than the IL-17 threshold (2), and as “having a risk” when the IL-17 level is less than the IL-17 threshold (1) or not less than the IL-17 threshold (2),

evaluating a risk of the subject with regard to the peptide vaccine by comparing an IL-1RA level in a blood sample from the subject with an IL-1RA threshold, and in the evaluation, an evaluation is given as “having no risk” when the IL-1RA level is less than the IL-1RA threshold, and as “having a risk” when IL-1RA level is not less than the IL-1RA threshold,

evaluating a risk of the subject with regard to the peptide vaccine by comparing a CCL4 level in a blood sample from the subject with a CCL4 threshold, and in the evaluation, an evaluation is given as “having a risk” when the CCL4 level is less than the CCL4 threshold, and as “having no risk” when the CCL4 level is not less than the CCL4 threshold, and

evaluating a risk of the subject with regard to the peptide vaccine evaluating by comparing an Hp level in a blood sample from the subject with an Hp threshold, and in the evaluation, an evaluation is given as “having a risk” when the Hp threshold includes an Hp threshold (1) and the Hp level is less than the Hp threshold (1); or an evaluation is given as “having no risk” when the Hp threshold includes an Hp (1) and an Hp threshold (2) that is greater than the Hp (1), and the Hp level is not less than the Hp threshold (1) and less than the Hp threshold (2), and as “having a risk” when the Hp level is less than the Hp threshold (1) or not less than the Hp threshold (2).

[Item 19] The determination method according to Item 18, wherein the subject is determined as an “ineligible person” for the peptide vaccine in the determination step when an evaluation is given as “having a risk” based on at least one prognostic factor.

[Item 20] The determination method according to Item 12, wherein the evaluation step based on a lymphocyte comprises any of the following evaluations depending on the lymphocyte selected:

evaluating a risk with regard to administration of the peptide vaccine by comparing a CD11b⁺CD14⁺HLA-DR^{low} immunosuppressive monocyte level in a blood sample from the subject with a corresponding threshold thereof, and in the evaluation, an evaluation is given as “having no risk” when the CD11b⁺CD14⁺HLA-DR^{low} immunosuppressive monocyte level is less than the corresponding threshold, and as “having a risk” when the CD11b⁺CD14⁺HLA-DR^{low} immunosuppressive monocyte level is not less than the corresponding threshold,

evaluating a risk with regard to administration of the peptide vaccine by comparing a CD3⁺CD4⁺CD45RA⁻T cell level in a blood sample from the subject with a corresponding threshold thereof, and in the evaluation, an evaluation is given as “having no risk” when the CD3⁺CD4⁺CD45RA⁻T cell is less than the corresponding threshold, and as “having a risk” when the CD3⁺CD4⁺CD45RA⁻ T cell is not less than the corresponding threshold, and

evaluating a risk with regard to administration of the peptide vaccine by comparing a CD4⁺CD25⁺FoxP3⁺ cell (Treg) level in a blood sample from the subject with a corresponding threshold thereof, and in the evaluation, an evaluation is given as “having no risk” when the CD4⁺CD25⁺FoxP3⁺ cell (Treg) is less than the corresponding threshold, and as “having a risk” when the CD4⁺CD25⁺FoxP3⁺ cell (Treg) is not less than the corresponding threshold.

[Item 21] The determination method according to Item 20, wherein administration of the peptide vaccine to the subject is determined to be “cancelled” in the determination step when an evaluation is given as “having a risk” based on at least one lymphocyte in a blood sample.

Examples are described below. These Examples merely show preferable embodiments of the present invention and should not be construed as limitation of the inventions outlined in the attached claims in any manner.

### EXAMPLES

### [Clinical Trial and Evaluation]

The test subjects were HLA-A24 positive patients suffering from a glioma resistant to temozolomide therapy. The test subjects were, under the treatment of Best Supportive Care (BSC), subjected to a double-blind comparative study in which an active-drug group was compared to a placebo group. This clinical trial was carried out in 20 medical facilities in Japan from 2011 to 2016 as a multicenter trial (phase-III clinical trial). The evaluation items included “overall survival (OS)” as a significant evaluation item, and included “12-month survival rate”, “tumor reduction effect”, “overall anti-tumor effect”, and “immunity” and “safety” as supplemental evaluation items. The “survival rate” refers to, in the Examples, a rate obtained by dividing the number of survived test subjects in a subject group at days elapsed from the initiation of the clinical study by the number of the test subjects in the subject group at the initiation time of the clinical trial. The days elapsed from the initiation of the clinical study correspond to their survival time.

### [Test Subjects Participated]

The test subjects were selected based on performance status (PS). The PS was proposed by the Eastern Cooperative Oncology Group (ECOG) in the United States and has been used in Japan, too. Ninety test subjects who had a PS of grade up to 3 regarding neurological symptoms were accepted and registered. Eighty-eight test subjects among them participated in the clinical trial.

### [Study Product]

An active drug (tailor-made peptide vaccine) was a tailor-made peptide vaccine that included at most 4 peptide antigens selected from the group consisting of 14 peptide antigens shown in Table 1 in descending order of immunoreactivities of the blood from glioma patients to each peptide antigen. As a result, Lck-208 and EZH2-735 among the 14 peptide antigens were not selected as an active drug in the clinical trial. The active drug in the trial accordingly included at most 4 peptides selected from the group consisting of SART2-93, SART3-109, PAP-213, PSA-248, EGF-R-800, MRP3-503, MRP3-1293, SART2-161, Lck-486, Lck-488, PSMA-624, and PTHrP-102 in descending order of the reactivity of the glioma patient's blood to each peptide antigen. The placebo was a suspension including an adjuvant alone in a solvent but not any peptide antigens. The peptides for an active drug used in the clinical trial were provided by Green Peptide Co., Ltd.

### [Dosing Regimen]

Among the 88 test subjects, 58 test subjects received a weekly administration of an active drug for about three months (hereinafter referred to also as “active-drug group”) and the remaining 30 test subjects received a weekly-administration of a placebo for about three months (hereinafter referred to also as “placebo group”). The test subjects were measured for the immunoreactivities of their blood after 12 times administrations of the study product over about three months. Peptide antigens to be included in an active drug were re-selected based on the results of the immunoreactivities. The newly-prepared active drug was biweekly administered six times, and then the peptide antigens to be included in an active drug were re-selected again.

### [Results of the Clinical Trial]

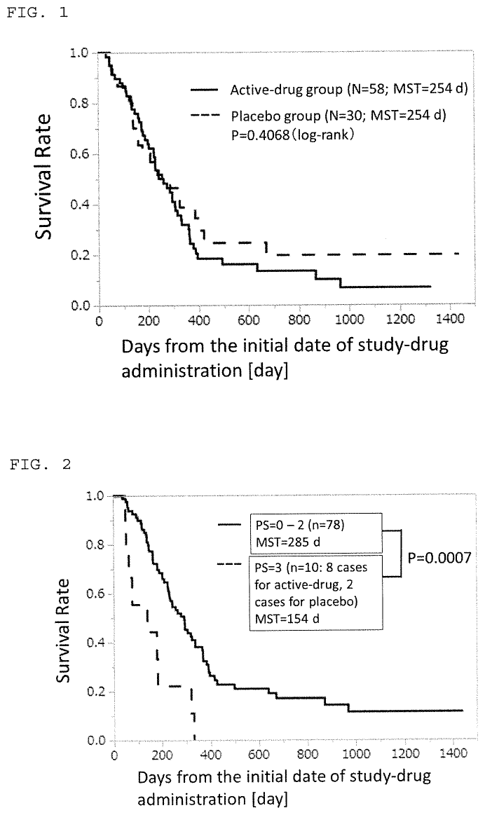

one serious adverse event (pulmonary embolism, grade 3), which could not deny a cause-and-effect relationship, occurred in the active-drug group. There were no serious adverse events, which could not deny a cause-and-effect relationship, except for the event above. The clinical trial revealed that the active-drug group showed the median survival time (MST) of the overall survival was 254 days, while the placebo group showed the MST of the overall survival was 254 days. The active drug was compared to the placebo for the survival rates versus the number of days elapsed from the administration to evaluate the efficacy of the active drug (FIG. 1). As a result, no statistically significant difference was found between the active-drug group (solid line) and the placebo group (broken line).

### [Subgroup Analysis (1)]

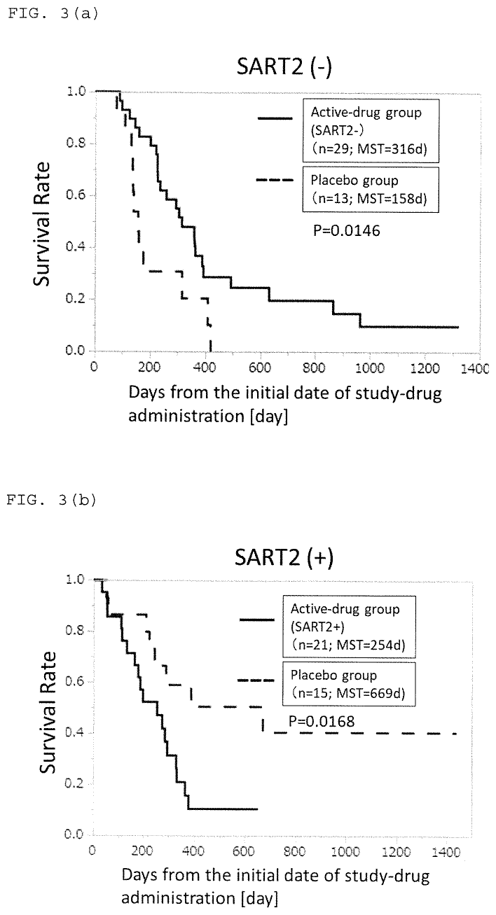

The subgroup analysis (1) was carried out to examine the results of the clinical trial for PS grades. As a result, test subjects having a PS of grade 3 showed the MST of the overall survival being 154 days, while test subjects having a PS of grade 0 to 2 showed the MST of the overall survival being 285 days. The survival rate versus the number of days elapsed from the administration of the study product was compared between the two groups (FIG. 2). As a result, the test subjects who had a PS of grade 3 showed a significantly shortened overall survival (broken line) than the test subjects who had a PS of grade 0 to 2 (solid line) (P<0.001).

For the test subjects having a PS of grade 0 to 2, a test subject group with a PS of grade 0, a test subject group with a PS of grade 1, and a test subject group with a PS of grade 2 were mutually compared for the survival rates. As a result, no significant differences were found in the overall survival between those test subject groups (not shown in the figures). The subgroup analysis (1) revealed that the health condition of a test subject having a PS of grade 3 significantly affects the survival time of the test subject. Test subjects who had a PS of grade 0 to 2 and were capable of doing daily activities were subjected to subgroup analyses further.

### [Subgroup Analysis (2)]

It is considered that a factor capable of predicting the prognosis of a glioma patient in response to an active drug enables us to determine in advance the eligibility of the glioma patient for the active drug. An additional subgroup analysis was accordingly carried out to find a further candidate factor. SART2 antigen was focused on as a candidate factor in consideration of the technical matter described below.

The SART2 antigen is known as an enzyme involved in proteoglycan synthesis (Masanobu Nakao, et al., J Immunol Mar. 1, 2000, 164 (5) 2565-2574) and is also called dermatan sulfate epimerase 1 (DS-epi1) (Marco Maccarana, et al., THE JOURNAL OF BIOLOGICAL CHEMISTRY VOL. 281, NO. 17, pp. 11560-11568). The SART2 antigen relates to the function of the brain or glioma. As to the functional relationship of SART2 with the brain or glioma, the followings are known: DS-epi1 (SART2) relates to productign iduronic acid A involved in production of chondroitin sulfate/dermatan sulfate proteoglycans (CS/DS-PGs) (Anders Malmstrom, et al., J Histochem Cytochem. 2012 December; 60 (12): 916-925); glycosylated chondroitin sulfate proteoglycans (CSPGs) prevent glioma from infiltrating by activating a tumor-associated microglia, and lack in diffusely infiltrating tumor cells (Daniel J. Silver, et al., Journal of Neuroscience 25 Sep. 2013, 33 (39) 15603-15617); DS deficiency but not CS deficiency suppresses the growth of neural stem cells and enhances expressions of the fibroblast growth factor 2 receptor (FGF-2R) and epidermal growth factor receptor (EGFR) (Shan Bian, et al., Journal of Cell Science 124, 4051-4063); SART2 is expressed in glioma but not in low grade astrocytoma (Antonio Bertolotto, et al., Neuro-Oncology 4: 43-48, 1986); and Endocan (a kind of DS, present also in the blood, attracts attention as a tumor marker) is found to be expressed in palisading cells which surround glioma like a fence and an Endocan-positive patient shows poor prognosis (Claude-Alain Maurage, et al., J Neuropathol Exp Neurol, Vol. 68, No. 6, June 2009, pp. 633Y641).

The relationship between the survival time of a test subject and the immunoreactivity to SART2 peptide was examined for whether SART2 antigen can be used as a prognostic factor. The active drug included at most 4 peptide antigens, which were selected from a peptide antigen group consisting of 14 peptide antigens shown in Table 1, in light of the immunoreactivities of a test subject to them. When the test subject has the blood showing a high immunoreactivity to SART2 peptide which is within the top 4, SART2 peptide is to be included in the peptide vaccine. The peptide antigen group consisting of 14 peptide antigens shown in Table 1 includes two types of SART2 peptide, i.e., SART2-93 peptide (SEQ ID No. 1) and SART2-161 peptide (SEQ ID No. 9) (see Table 1). The subgroup analysis (2) was carried out to examine the clinical trial results of the test subjects having a PS of grade 0 to 2 for the relationship between the survival times and whether or not at least one SART2 peptide, namely SART2-93 peptide (SEQ ID No. 1) or SART2-161 peptide (SEQ ID No. 9), was selected as a component of the active drug, as a substitute for examining the relationship between the survival times and the immunoreactivities of the test subjects to the SART2 peptide.

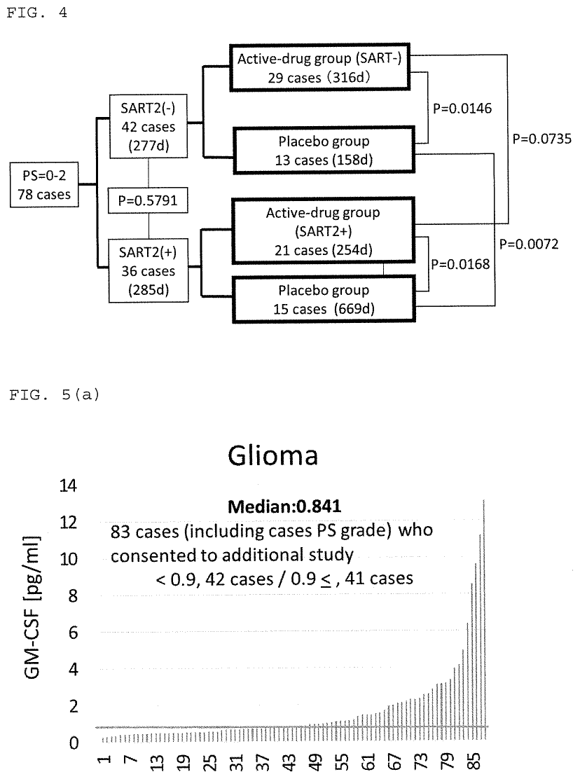

There were 42 test subjects for whom SART2 peptide was not selected as a component of the active drug (SART2−) (FIG. 3a). Among the 42 test subject, 29 test subjects belonged to the active-drug group and the MST of the overall survival was 316 days. And 13 test subjects belonged to the placebo group and the MST of the overall survival was 158 days. The survival rate versus the number of days elapsed after the administration of the study product was compared between the two groups (FIG. 3a). As a result, the active-drug group showed a significantly extended overall survival (solid line) than the placebo group (broken line) (P=0.0146, log-rank test: unless otherwise specified, the log-rank test will also be used hereinafter as a test method).

Besides, there were 36 test subjects for whom SART2 peptide was selected as a component of the active drug (SART2+) (FIG. 3b). Among the 36 test subjects, 21 test subjects belonged to the active-drug group and the MST of the overall survival was 254 days. And 15 test subjects belonged to the placebo group and the MST of the overall survival was 669 days. The survival rate versus the number of days elapsed after the administration of the study product was compared between the two groups (FIG. 3b). As a result, the active-drug group showed a significantly shortened overall survival (solid line) than the placebo group (broken line) (P=0.0168).

The subgroup analysis (2) revealed that the immunoreactivity of a test subject to SART2 peptide before the administration of the study product affects its survival time in the active-drug group compared to that in the placebo group. The result suggests that the immunoreactivity of a glioma patient to SART2 peptide can be used as a prognostic factor for determining in advance the eligibility of the glioma patient for the active drug, and further for determining in advance an effect of the active drug on the glioma patient.

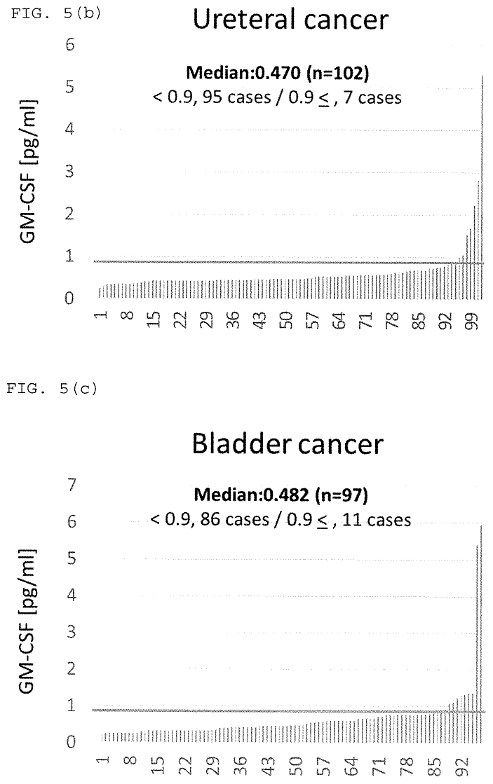

FIG. 4 schematically shows the results of the subgroup analysis (2) in which the 78 test subjects who had a PS of grade 0 to 2 were assessed for whether SART2 peptide was selected as a component of the active drug based on the immunoreactivities of the test subjects' blood to the SART2 peptide before the administration of the active drug or the placebo.

### [Subgroup Analysis (3)]

An additional subgroup analysis was carried out to find a further candidate factor for determining in advance the eligibility of a glioma patient for an active drug. Cytokine GM-CSF was focused on as a candidate factor in consideration of the technical matters involved in GM-CSF (described below) and concentrations of the cytokine in the blood from several types of cancer patients.

TGM-CSF is known as a cytokine that facilitates differentiation of pluripotent hematopoietic stem cells and as being involved in the proliferation of microglia cells, which are also called brain macrophages, as well as are known as being produced by glioma cells and involved in the growth of themselves (Margareta M. Mueller, et al., American Journal of Pathology, Vol. 155, No. 5, November 1999). GM-CSF facilitates the activation of T helper 1 (Th1) cells and is accordingly frequently used as an adjuvant in the field of cancer vaccines. Many documents report that GM-CSF was used in a combination therapy for early cancer and for preventing a recurrence (Christoph Hoeller, et al., Immunol Immunother (2016) 65: 1015-1034 and G. Parmianil, et al., Annals of Oncology 18: 226-232, 2007). Further, it is reported that a high blood GM-CSF concentration led to a good prognosis for lung cancer patients and for esophageal cancer patients, and reported that the administration of GM-CSF or the immunotherapy with the addition of GM-CSF enhanced the immunity led to a good prognosis (Christoph Hoeller, et al., Cancer Immunol Immunother (2016) 65: 1015-1034; G. Parmianil, et al., Annals of Oncology 18: 226-232, 2007; and Guodong Deng, et al., Oncotarget, 2016, Vol. 7, (No. 51), pp: 85142-85150).

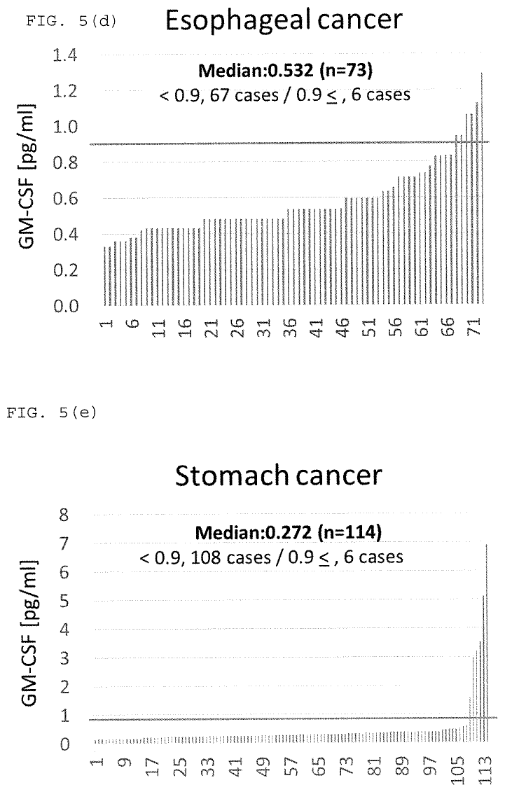

The blood GM-CSF concentrations were measured in a glioma group of 83 patients (including the test subjects with a PS of grade 3) who signed a consent form of additional studies, besides in a urinary cancer group of 102 patients, in a bladder cancer group of 97 patients, in an esophageal cancer group of 73 patients, in a gastric cancer group of 114 patients, and in a biliary tract cancer group of 101 patients (FIG. 5). As a result, the glioma group showed the median value of GM-CSF concentrations was 0.841 (FIG. 5a); the ureteral cancer group showed 0.470 (FIG. 5b); the bladder cancer group showed 0.482 (FIG. 5c); the esophageal cancer group showed 0.532 (FIG. 5d); the stomach cancer group showed 0.272 (FIG. 5e); and the biliary tract cancer group showed 0.364 (FIG. 5f). The glioma group showed a significantly higher median value of GM-CSF concentrations than the other carcinoma groups (all, P<0.001). These results suggest that cytokine GM-CSF possibly involves in growth suppression of cancer cells, particularly in glioma patients.

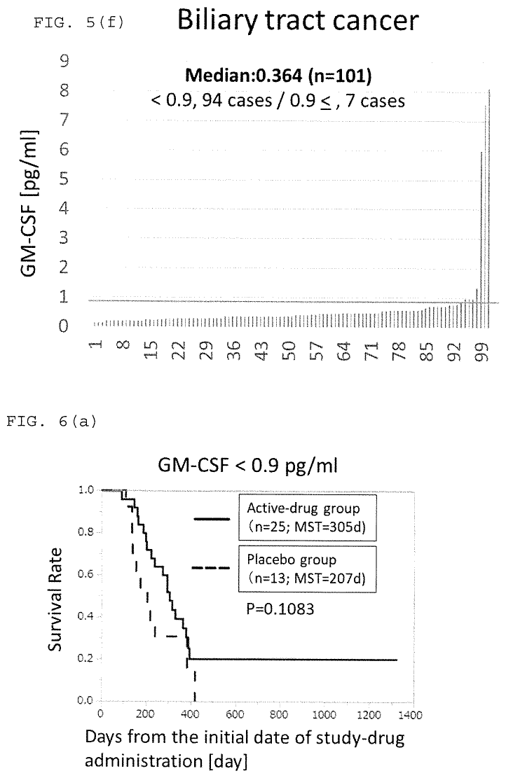

The subgroup analysis (3) examined the clinical trial results of the test subjects who had a PS of grade 0 to 2 (75 test subjects excluding 3 test subjects who did not sign a consent form for additional studies) for the relationship between the survival time and the blood GM-CSF concentration. The clinical trial results of the test subjects were divided into two groups based on an indicator, namely whether or not the blood GM-CSF concentration of the test subject before the administration of the study product was less than 0.9 pg/mL. The number of the test subjects who had the GM-CSF concentration of less than 0.9 pg/mL was 38 (FIG. 6a), while the number of the test subjects who had the GM-CSF concentration of not less than 0.9 pg/mL was 37 (FIG. 6b).

There were 38 test subjects who had the GM-CSF concentration of less than 0.9 pg/mL. Among the 38 test subjects, 25 test subjects belonged to the active-drug group and 13 test subjects belonged to the placebo group. The active-drug group showed the MST of the overall survival was 305 days, while the placebo group showed the MST of the overall survival was 207 days. The survival rate versus the number of days elapsed after the administration of the study product was compared between the two groups (FIG. 6a). As a result, no significant difference was found in overall survival between the active-drug group (solid line) and the placebo group (broken line) (P=0.1083). However, the survival time of the active-drug group was found to tend to extend.

Also, there were 37 test subjects who had the GM-CSF concentration of not less than 0.9 pg/mL. Among the 37 test subjects, 22 test subjects belonged to the active-drug group and 15 test subjects belonged to the placebo group. The active-drug group showed the MST of the overall survival was 227.5 days, while the placebo group showed the MST of the overall survival was 410 days. The survival rate versus the number of days elapsed after the administration of the study product was compared between the two groups (FIG. 6b). As a result, the active-drug group showed a significantly shortened overall survival (solid line) than the placebo group (broken line) (P=0.0180).

The subgroup analysis (3) revealed that the blood GM-SCF concentration of a test subject before the administration of a study product affects its survival time in an active-drug group as compared to that in the placebo group. The result suggests that GM-CSF can be used as a prognostic factor for determining in advance the eligibility of a glioma patient for an active drug, and further for predicting an effect of the active drug on the glioma patient.

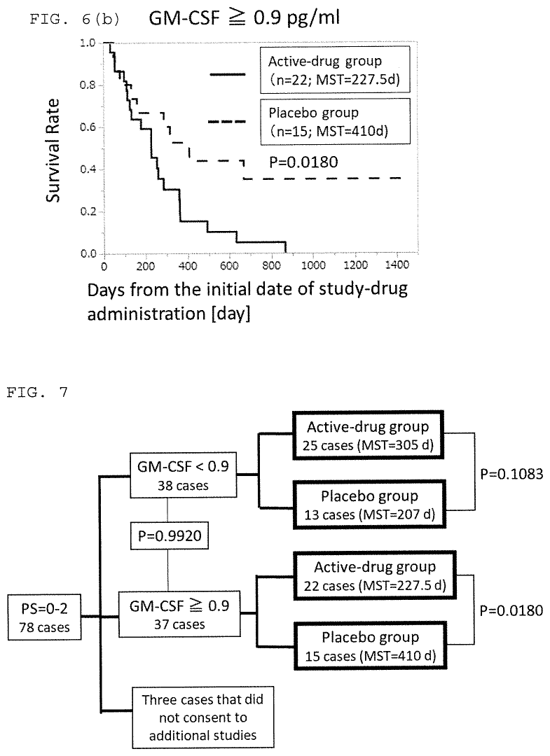

FIG. 7 schematically shows the results of the subgroup analysis (3) in which the 75 test subjects who had a PS of grade 0 to 2 were assessed based on the blood GM-CSF concentrations of the test subjects before the administration of the active drug or the placebo.

### [Subgroup Analysis (4)]

A subgroup analysis (4) was carried out to assess a combination of two candidate factors for whether the eligibility of a test subject for an active drug can be determined in advance or not. The two candidate factors were the blood GM-CSF concentration that was suggested as a prognostic factor for a glioma patient and the immunoreactivity to SART2. The subgroup analysis (4) examined the clinical test results of the test subjects having a PS of grade 0 to 2 (77 test subjects excluding one test subject who did not sign a consent form for additional studies) for the relationship between the survival time and the blood GM-CSF concentration of 0.9 pg/mL or whether the SART2 peptide was selected as a component of the active drug.

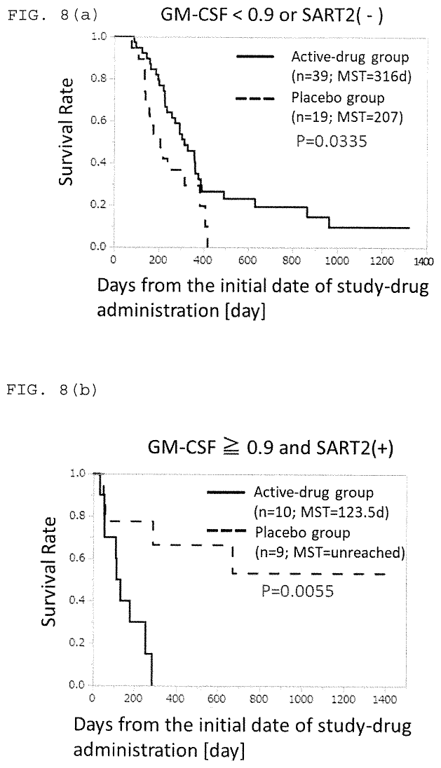

There were 58 test subjects who satisfied either the blood GM-CSF concentration was less than 0.9 pg/mL before the administration of the study product or the SART2 peptide was not selected as a component of the active drug (SART2−) based on the immunoreactivity of the blood from the test subjects (FIG. 8a). Among the 58 test subjects, 39 test subjects belonged to the active-drug group and the MST of the overall survival was 316 days. And 19 test subjects belonged to the placebo group and the MST of the overall survival of 207 days. The survival rate versus the number of days elapsed after the administration of the study product was compared between the two groups (FIG. 8a). As a result, the active-drug group showed a significantly extended overall survival (solid line) than the placebo group (broken line) (P=0.0335).

There were 19 test subjects who satisfied both the blood GM-CSF concentration was not less than 0.9 pg/mL before the administration of the study product and the SART2 peptide was selected as a component of the active drug (SART2+) based on the immunoreactivity of the blood from the subjects (FIG. 8b). Among the 19 test subjects, 10 test subjects belonged to the active-drug group and the MST of the overall survival was 123.5 days. And 9 test subjects belonged to the placebo group and the MST of the overall survival was “unreachable”. The survival rate versus the number of days elapsed after the administration of the study product was compared between the two groups (FIG. 8b). As a result, the active-drug group showed a significantly shortened overall survival (solid line) than the placebo group (broken line) (P=0.0055).

The subgroup analysis (4) revealed that the combination of the two factors, namely the blood GM-CSF concentration of a test subject before the administration of the study product and the immunoreactivity to the SART2 peptide, affects the survival time in the active-drug group as compared to that in the placebo group. The result suggests that the combination of the two factors can be used as a prognostic factor for determining in advance the eligibility of a glioma patient for an active drug, and further for predicting in advance an effect of the active drug on the glioma patient.

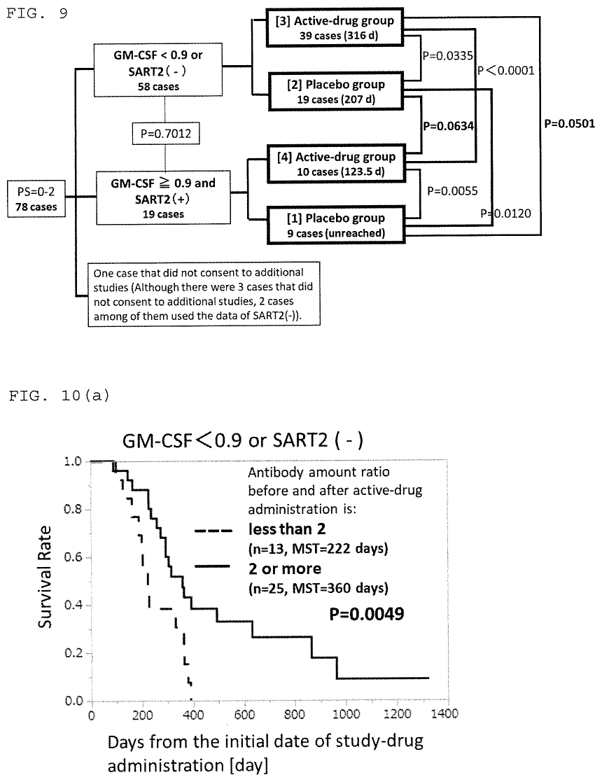

FIG. 9 schematically shows the results of the subgroup analysis (4) in which the 77 test subjects who had a PS of grade 0 to 2 were assessed for the combination of the two factors, SART2 and GM-CSF. As seen from FIG. 9, the active drug was effective for the patients who satisfied either the blood GM-CSF concentration was less than 0.9 pg/mL or the SART2 peptide was not selected as a component of the active drug (SART2−) (FIG. 9, the active-drug group [3] showed a significantly extended overall survival compared to the placebo group [2] (P=0.0335)). The clinical test revealed that the active drug was effective for 39 test subjects (FIG. 9, the active-drug group [3] (n=39)) among the 58 test subjects in the active group (see the above section “Dosing regimen”) (67%).

On the other hand, the combination of two factors, namely the blood GM-CSF concentration and the immunoreactivity to the SART2 peptide of a glioma patient, can be used to determine in advance the ineligibility of the glioma patient for an active drug. Such an advance determination of ineligibility can provide the 10 test subjects in the active-drug group [4] in FIG. 9 with, for example, a benefit of avoiding immunotherapy using the active drug and can provide them with an opportunity to receive another treatment. An additional benefit in light of medical economics is to lead to saving a cost involved in the preparation or the administration of active drugs (tailor-made peptide vaccine).

### [Immune-State Analysis (1)]

As shown in FIG. 9, the placebo group [1], which satisfied both the blood GM-CSF concentration of a test subject was not less than 0.9 pg/mL before the administration of the active drug or the placebo and the SART2 peptide was selected as a component of the active drug (SART2+), showed that the overall survival was “unreachable” (n=9), while the placebo group [2], which satisfied either the GM-CSF concentration is less than 0.9 pg/mL and or SART2 peptide was not selected as a component of the active drug (SART2−), showed that the MST of the overall survival was 207 days (n=19). The two placebo groups showed significantly different overall survival (P=0.012).

The two placebo groups were divided based on the blood cytokine GM-CSF concentration of the test subjects and the immunoreactivities to SART2 peptide before the administration of the study product. Accordingly, the significant difference in overall survival between the two groups should be irrelevant to the administration of the study product. The difference in overall survival may have been caused by a difference in the immune state of test subjects associated with or involved in the GM-CSF level.

An immune-state analysis (1) was accordingly carried out to examine the test subjects of the two placebo groups for the 34 cytokines' states. The result showed that the test subjects in the placebo group [1] had higher concentrations of 34 cytokines except three cytokines (BAFF, haptoglobin, and eotaxin) than the test subjects in the placebo group [2] (data not shown). The immune-state analysis (1) suggests that the immune state (cytokine concentration) of a test subject affects its survival time.

### [Immune-State Analysis (2)]

The subgroup analysis (4) showed that the active-drug group [3] showed a significantly extended overall survival (MST: 316 days) than the placebo group [2] (MST: 207 days) (P=0.0335). The result suggests that the active drug was effective. The active-drug group [4] showed a significantly shortened overall survival (MST: 123.5 days) than the placebo group [1] (MST: unreachable) (P=0.0055). The result suggests that the active drug was ineffective.

The active-drug group [3] and the active-drug group [4] were divided based on the blood cytokine GM-SCF concentration and the immunoreactivity to SART2 peptide before the administration of the active drug. Accordingly, the results that the active drug was effective in one group (active-drug group [3]) and was ineffective in the other group (active-drug group [4]) may have been caused by a difference in the immune state of test subjects associated with or involved in cytokine GM-CSF level, in the light of the results of immune-state analysis (1).

An immune-state analysis (2) was then carried out to examine the cytokine states of the test subjects in the two active-drug groups. The result showed that the test subjects in the active-drug group [3] had lower concentrations of 22 cytokines than the test subjects in the active-drug group [4]. Particularly, the test subjects in the active-drug group [3] had lower concentrations of GM-CSF and cytokines involved in downstream signaling pathways than the active-drug group [4] (data not shown). The immune-state analysis (2) also suggests that the immune state (cytokine concentration) of a test subject affects its survival time.

### [Immune-State Analysis (3)]

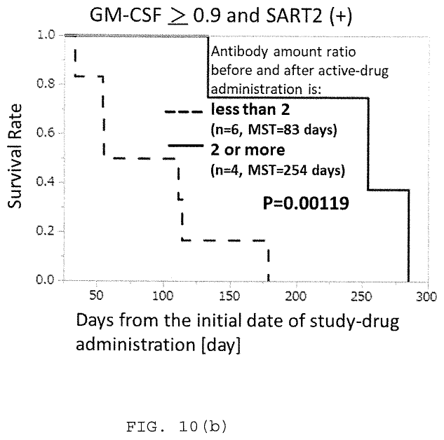

Further to examine the immune states of the test subjects in the active-drug groups [3] and [4] in FIG. 9, an immune-state analysis (3) was carried out to measure the amount of an antibody in the blood from the individual test subjects before and after the administration of the active drug. There were 39 test subjects in the active-drug group [3], who had the GM-CSF concentration of less than 0.9 or for whom the SART2 peptide was not selected as a component (SART2−). A ratio of blood antibody amount before the administration of an active drug to that after the administration of the active drug (=[blood antibody amount after the administration of an active drug]/[blood antibody amount before administration of the active drug]) was measured for 38 out of the 39 test subjects (FIG. 10a). Among the 38 test subjects, 13 test subjects showed the antibody amount ratio of less than two and the MST of the overall survival was 222 days. And 25 test subjects showed the antibody amount ratio of not less than two and the MST of the overall survival was 360 days. Regarding the survival rate versus the number of days elapsed from the administration of the study product, the test subjects who showed the antibody amount ratio of not less than two were compared with the test subjects who showed the antibody amount ratio of less than two (FIG. 10a). As a result, the test subjects who showed the antibody amount ratio of not less than two showed a significantly extended overall survival (solid line) than the test subjects who had an antibody amount ratio of less than two (broken line) (P=0.0049).

The active-drug group [4], in which the GM-CSF concentration was not less than 0.9 pg/mL and the SART2 peptide was selected as a component (FIG. 10b), included 10 test subjects. Among the 10 test subject, 6 test subjects who showed the antibody amount ratio of less than two showed the MST of the overall survival was 83 days. And 4 test subjects who showed the antibody amount ratio of not less than two showed the MST of the overall survival was 254 days. Regarding the survival rate versus the number of days elapsed from the administration of the study product, the test subjects who showed the antibody amount ratio of not less than two were compared with the test subjects who showed the antibody amount ratio of less than two (FIG. 10b). As a result, the test subjects who showed the antibody amount ratio of not less than two showed a significantly extended overall survival (solid line) than the test subjects who showed the antibody amount ratio of less than two (broken line) (P=0.0119). The immune-state analysis (3) suggests that the antibody amount in the blood of a test subject increases by the administration of the active drug, leading to the activation of the immune state involved in the cancer, and affecting its survival time.

### [Immune-State Analysis (4)]

The immune-state analysis (1) to (3) suggested that the immune state of a test subject, particularly the cytokine levels, affects an effect of the immunotherapy using an active drug (i.e., survival time). An immune-state analysis (4) accordingly focused on a kind of cytokines, namely MCP-1, as a candidate factor of determining in advance the eligibility of a glioma patient for an active drug and examined the MCP-1 level of each of the groups [1] to [4] in FIG. 9.

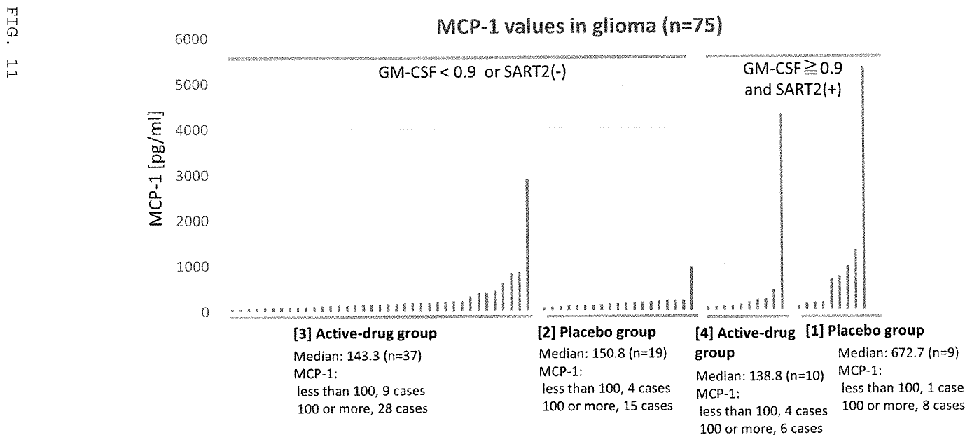

The cytokine levels in the blood in FIG. 9 should not be largely different between 10 test subjects in the active-drug group [4] and 9 test subjects in the placebo group [1] because the levels were measured before the administration of the study product (the active drug or the placebo). However, the active-drug group [4] showed a lower cytokine MCP-1 level than the placebo group [1], in particular the active-drug group [4] showed 138.8 as the median value of the cytokine MCP-1 level (FIG. 11, active-drug group [4]), while the placebo group [1] showed 672.7 as the median value (FIG. 11, placebo group [1]). The difference in MCP-1 level between the two groups is considered as being caused by a bias that the number of cases was small. However, the difference in MCP-1 level was presumed to cause the significant difference in overall survival between the active-drug group [4] (MST=123.5) and the placebo group [1] (unreachable). The presumption would be considered proper in light of the technical matter involved in MCP-1 described below.

MCP-1 is one of the C—C chemokine family that primarily exhibits chemotaxis to monocytes (also called as chemokine (C—C motif) ligand 2, CCL2). MCP-1 is a chemokine produced in monocytes, vascular endothelial cells, and fibroblasts, and is regarded as being indispensable for immune cells such as monocytes, memory T cells, and dendritic cells, as migrating to inflammatory sites such as cancer sites. MCP-1 is found to play an important role in glioma (Alireza Vakilian, et al., Neurochemistry International 103 (2017)). Since MCP-1 strongly induces inhibitory T cells (Treg) (Chiara Vasco, et al., J Neurooncol (2013) 115: 353-363; Justin T. Jordan, et al., Cancer Immunol Immunother (2008) 57: 123-131; and Xin Chen, et al., Int Immunopharmacol. 2016 May; 34: 244-9), blocking the CCL2 pathway is suggested as being able to be effective on cancer immunotherapy (Zvi G. Fridlender, et al., Cancer Res; 70 (1); 109-18).

Treg is also reported to be suppressed by treating glioma with an anticancer agent (Justin T. Jordan, et al., Cancer Immunol Immunother (2008) 57: 123-131). An anti-cancer agent such as cyclophosphamide, which is the same alkylating agent as glioma therapeutic drug, temozolomide, is known to suppress Treg and to enhance a cancer immune effect (Madondo M T, et al., Cancer Treat Rev 2016; 42: 3-9; and Abu Eid R, et al., Cancer Immunol Res 2016; 4: 377-82).

### [Subgroup Analysis (5)]

A subgroup analysis (5) was carried out to assess whether the cytokine MCP-1, which was suggested as being available as a prognostic factor in the immune-state analysis (4), can determine in advance the eligibility of a patient for an active drug. In the subgroup analysis (5), the clinical trial results of test subjects who had a PS of grade 0 to 2 (75 test subjects excluding 3 test subjects who did not sign a consent form for additional studies) were examined for the relationship between the survival time and the blood MCP-1 concentration of test subjects before the administration of a study product.

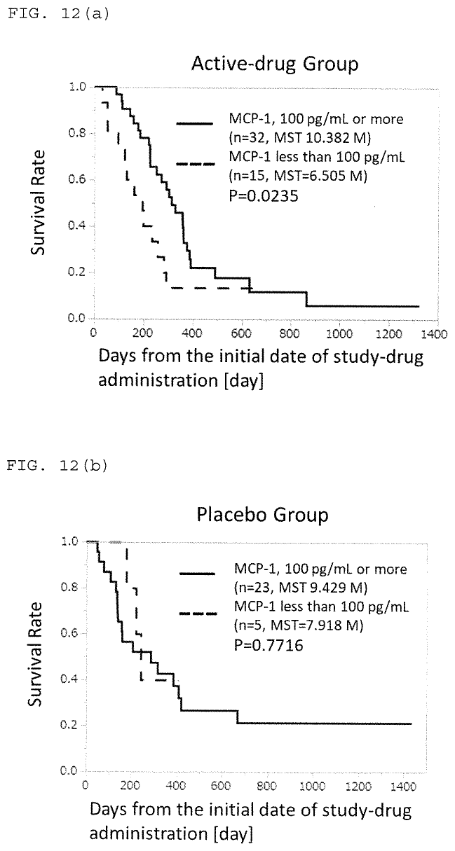

The subgroup analysis (5) divided into two groups the clinical trial results of the total 28 test subjects that included 9 test subjects in the placebo group [1] and 19 test subjects in the placebo group [2] in FIG. 9 (FIG. 12b). One group included 23 test subjects having the blood MCP-1 concentration of not less than 100 pg/mL and showed the MST of the overall survival was 9.429 months (solid line). The other group included 5 test subjects having the blood MCP-1 concentration of less than 100 pg/mL and showed the MST of the overall survival was 7.918 months (broken line). The survival rate versus the number of days elapsed after placebo administration was compared between the two groups (FIG. 12b). As a result, no significant difference was found in survival time between the test subject group that showed the MCP-1 of not less than 100 pg/mL (solid line) and the test subject group that showed the MCP-1 of less than 100 pg/mL (broken line) (P=0.7716).

Besides, 47 test subjects in the active-drug group, which included the active-drug group [3] (n=37) and the active-drug group [4] (n=10) (FIG. 9), were divided into two groups according to the index above (FIG. 12a). Among the 47 test subjects, 32 test subjects in the active-drug group, who had the blood MCP-1 concentration of not less than 100 pg/mL, showed the MST of the overall survival was 10.382 months. And 15 test subjects who had the blood MCP-1 concentration of less than 100 pg/mL showed the MST of the overall survival was 6.505 months. The survival rate versus the number of days elapsed after the administration of the active drug was compared between the two groups (FIG. 12a). As a result, the group of the test subjects who had the MCP-1 concentration of not less than 100 pg/mL showed a significantly extended overall survival (solid line) than the group of the test subjects who had the MCP-1 concentration of less than 100 pg/mL (broken line) (P=0.0235).

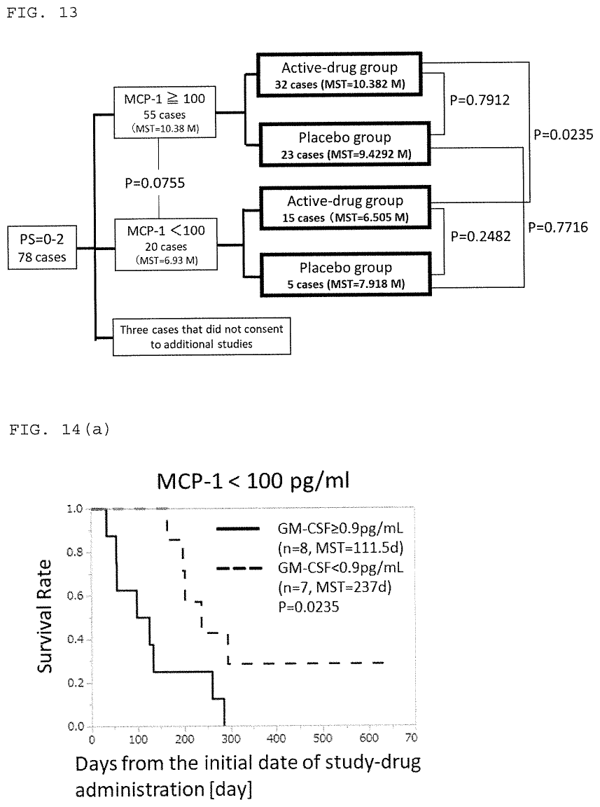

The subgroup analysis (5) revealed that the blood MCP-1 concentration of a test subject before the administration of an active drug affects the effect of an active drug on the glioma patient (i.e., survival time). This suggests that the blood MCP-1 concentration can be used as a prognostic factor for determining in advance the eligibility of a glioma patient to an active drug. FIG. 13 schematically shows the results of subgroup analysis (5) in which the 75 test subjects who had a PS of grade 0 to 2 were assessed for the blood MCP-1 concentration before the administration of the active drug or the placebo.

### [Subgroup Analysis (6)]

MCP-1 was suggested as being able to be used as a factor of determining in advance the eligibility of a glioma patient for an active drug. However, a plurality of factors are conceivable as prognostic factors for glioma in consideration of the diversity of glioma (Jennifer S. Sims, et al., J Neurooncol. 2015 July; 123 (3): 359-72; Jinquan Cai, et al., PLoS One. 2015 May 15; 10 (5): e0126022; and Mathieu Rodero, et al., J Clin Oncol. 2008 Dec. 20; 26 (36): 5957-64). For example, CX3CR1 genetic polymorphism (Mathieu Rodero, et al., J Clin Oncol. 2008 Dec. 20; 26 (36): 5957-64) and cytokines such as MCP-1, CCR2, CXCL10, IL17R, IL17B and IL10RB (Jinquan Cai, et al., PLoS One. 2015 May15; 10 (5): e0126022) are known as a prognostic factor of glioma. However, these cytokine factors, if used alone, are reported as not being able to use as a prognostic factor.

A subgroup analysis (6) was accordingly carried out to assess whether a combination of two factors, e.g., the blood MCP-1 concentration as one candidate and the blood GM-CSF concentration or the immunoreactivity to SART2 peptide as the other candidate, enables us to determine in advance the eligibility of the test subjects for an active drug. In the subgroup analysis (6), the clinical trial results of the 47 test subjects who had a PS of grade 0 to 2 in the active-drug group were examined for the relationship between the survival time and a combination of the factors.

An indicator of the blood MCP-1 concentration of a test subject was whether the blood concentration before the administration of the study product was not less than 100 pg/mL or less than 100 pg/mL. An indicator of the immunoreactivity to SART2 peptide was whether the peptide was selected as a component of the active drug or not, i.e., (SART2+) or (SART2−). An indicator of the blood GM-CSF concentration of a test subject was whether the blood concentration before the administration of the study product was not less than 0.9 pg/mL or less than 0.9 pg/mL.

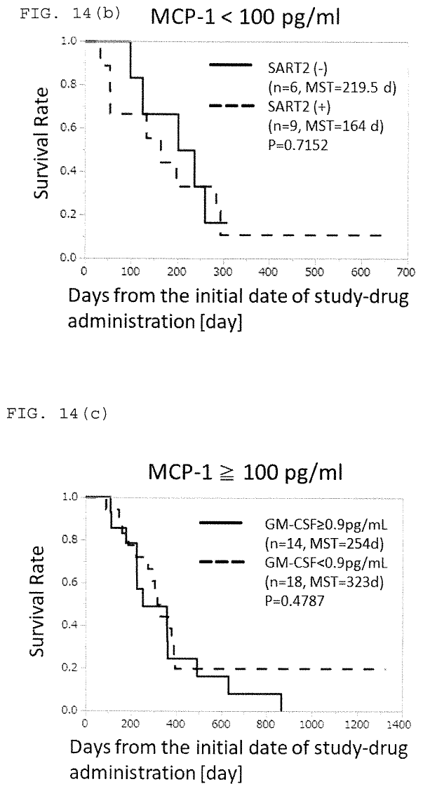

The active-drug group in which the MCP-1 concentration was less than 100 pg/mL included 15 test subjects (FIG. 14a, FIG. 14b), while the active-drug group in which the MCP-1 concentration was not less than 100 pg/mL included 32 test subjects (FIG. 14c, FIG. 14d). Among the 15 test subjects who had the MCP-1 concentration of less than 100 pg/mL in the active-drug group (FIG. 14a), 8 test subjects had the GM-CSF concentration of not less than 0.9 pg/mL before the administration of the active drug and showed the MST of the overall survival was 111.5 days. And 7 test subjects had the GM-CSF concentration of less than 0.9 pg/mL and showed the MST of the overall survival was 237 days. The survival rate versus the number of days elapsed after the administration of the active drug was compared between the two groups (FIG. 14a). As a result, the active-drug group that had the MCP-1 concentration of less than 100 pg/mL and the GM-CSF concentration of not less than 0.9 pg/mL showed a significantly shortened overall survival (solid line) than the active-drug group that had the GM-CSF concentration of less than 0.9 pg/mL (broken line) (P=0.0235). These accordingly suggest that the combination of the two factors, namely the blood GM-CSF concentration and the blood MCP-1 concentration less than a predetermined threshold (for example, 100 pg/mL), can be used as a prognostic factor for determining in advance the eligibility of a glioma patient for an active drug.

Among the 15 test subjects who had the MCP-1 concentration of less than 100 pg/mL in the active-drug group (FIG. 14b), 9 test subjects for whom SART2 peptide was selected as a component of the active drug (SART2+) showed the MST of the overall survival was 164 days. And 6 test subjects for whom SART2 peptide was not selected as a component of the active drug (SART2−) showed the MST of the overall survival was 219.5 days. The survival rate versus the number of days elapsed after the administration of the active drug was compared between the two groups (FIG. 14b). As a result, no significant difference was found in overall survival between the two groups (P=0.7152). Accordingly, it was not recognized that the combination of the two factors, namely the immunoreactivity to SART2 peptide and the blood MCP-1 concentration of less than a predetermined threshold (for example, 100 pg/mL), can be used for determining in advance the eligibility of a glioma patient for an active drug under the indexes used in this analysis.

Among the 32 test subjects who had the GM-CSF concentration of not less than 0.9 pg/mL before the administration of the active drug (FIG. 14c), 14 test subjects had the MCP-1 concentration of not less than 100 pg/mL before the administration of the active drug and showed the MST of the overall survival was 254 days. And 18 test subjects had the GM-CSF concentration of less than 0.9 pg/mL and showed the MST of the overall survival was 323 days. The survival rate versus the number of days elapsed after the administration of the active drug was compared between the two groups (FIG. 14c). As a result, no significant difference was found in overall survival between the two groups (P=0.4787). Accordingly, it was not recognized that the combination of the two factors, namely the blood GM-CSF concentration and the blood MCP-1 concentration of not less than a predetermined threshold (for example, 100 pg/mL), can be used for determining in advance the eligibility of a glioma patient for an active drug under the indexes used in this analysis.

Among the 32 test subjects had the MCP-1 concentration of not less than 100 pg/mL (FIG. 14d), 11 test subjects for whom SART2 peptide was selected as a component of the active drug (SART2+) showed the MST of the overall survival was 276 days. And 21 test subjects for whom SART2 peptide was not selected as a component of the active drug (SART2−) showed the MST of the overall survival was 360 days. The survival rate versus the number of days elapsed after the administration of the active drug was compared between the two groups (FIG. 14d). As a result, no significant difference was found in overall survival between the two groups (P=0.2372). Accordingly, it was not recognized that the combination of the two factors, namely the immunoreactivity to SART2 peptide and the blood MCP-1 concentration of not less than a predetermined threshold (for example, 100 pg/mL), can be used for determining in advance the eligibility of a glioma patient for an active drug under the indexes used in this analysis.

The subgroup analysis (6) revealed that the combination of the two factors, namely the blood MCP-1 concentration being less than a predetermined threshold of a test subject before the administration of an active drug and a blood GM-CSF concentration, affects the effect (i.e., survival time) of an active drug on a glioma patient. The combination of the two factors is suggested as being able to use for determining in advance the eligibility of a glioma patient for an active drug. The suggestion would be considered proper in light of the technical matters regarding the roles of MCP-1 and GM-CSF in the migration of immune cells described below.

The cytokines MCP-1 and GM-CSF are involved in the migration of immune cells. GM-CSF plays a role of activating monocytes, dendritic cells, memory T cells, and the like in the lymph node to which GM-CSF belongs. MCP-1 is responsible for the migration of activated immune cells to cancer sites, damaged sites, or infection sites. The immune cells activated by GM-CSF enter in blood flow and are circulated. When the immune cells reach cancer sites, damaged sites, or infection sites, the immune cells remove cancer cells at the sites, heal damages or control infection. The chemokine MCP-1 is required for the migration of the immune cells to cancer sites, damaged sites, or infection sites. MCP-1 attaches to the vascular endothelial cell surface and is involved in a mechanism of the migration of activated immune cells across the blood vessel cells out of the blood vessel. As described, GM-CSF is involved in the activation of immune cells to remove foreign substances and in the migration of activated immune cells.

### [Subgroup Analysis (7)]

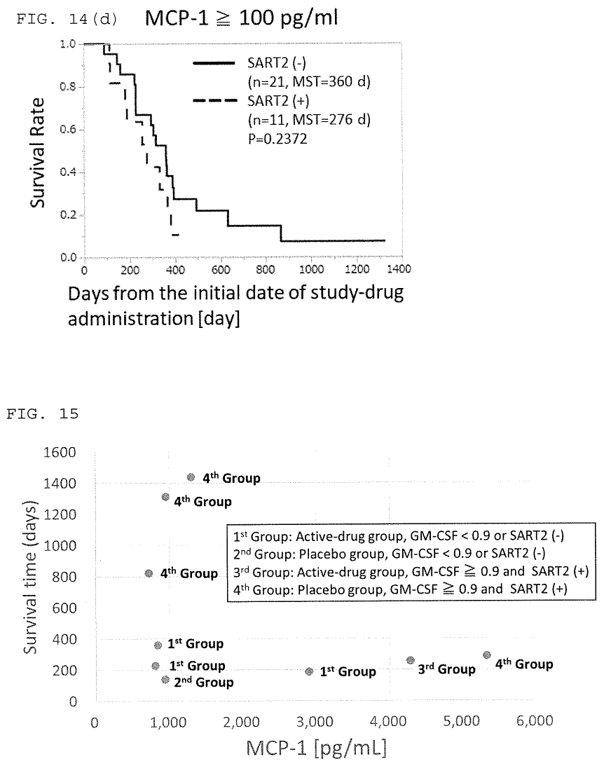

The subgroup analysis (6) revealed that, although no significant difference was found in overall survival between the two groups made by dividing the test subjects of the active-drug group based on the blood MCP-1 concentration of not less than a predetermined threshold (for example, 100 pg/mL) and on the immunoreactivity to SART2 peptide (FIG. 14d), the overall survival tended to differ (P=0.2372). In addition to the combination of the blood MCP-1 concentration of not less than 100 pg/mL and the immunoreactivity to SART2 peptide, a candidate factor blood GM-CSF concentration was combined and the obtained combination of the three factors was examined. More specifically, the relationship between the MCP-1 of not less than 700 pg/mL and the overall survival of each group below was examined (FIG. 15): first group; the active-drug group showing the GM-CSF concentration of less than 0.9 pg/mL or for which the SART2 peptide was not selected as a component of the active drug (SART2−) (corresponding to the active-drug group [3] in FIG. 9), second group; the placebo group satisfying the same condition as the first group (corresponding to the placebo group [2] in FIG. 9), third group; the active-drug group showing the GM-CSF concentration of not less than 0.9 pg/mL and the SART2 peptide was selected as a component as the active drug (SART2+) (corresponding to the active-drug group [4] in FIG. 9), and fourth group; the placebo group satisfying the same condition as the third group (corresponding to the placebo group [1] in FIG. 9).

As a result, the overall survival of the fourth group (placebo group) was as good as more than 800 days at MCP-1 beyond 700 pg/mL, while the overall survival of the first and third groups (active-drug groups) was as low as less than 400 days. The results suggest that a second threshold (e.g., 700 pg/mL) of the blood MCP-1 concentration can be used as a candidate factor in addition to the first threshold (e.g., 100 pg/mL) found in the subgroup analysis (6).

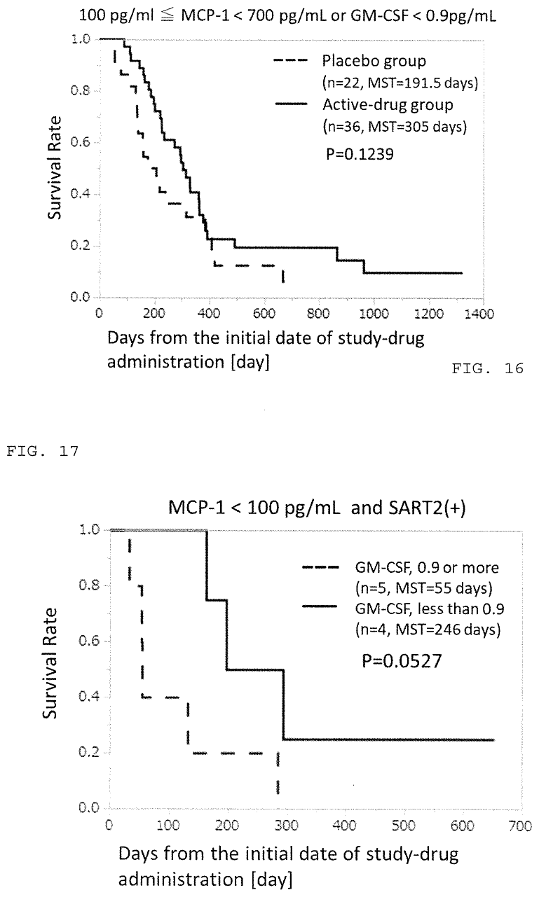

A subgroup analysis (7) was carried out to assess whether a combination of two factors, namely the blood MCP-1 concentration within a predetermined range and the blood GM-CSF concentration, can be used for determining in advance the eligibility of a test subject for an active drug. A predetermined range of from 100 pg/mL to 700 pg/mL for the blood MCP-1 concentration was used as an indicator. The blood GM-CSF concentration at 0.9 pg/mL was used as an indicator. There were 58 test subjects who had the blood MCP-1 concentration within the above-predetermined range or the blood GM-CSF concentration of less than 0.9 pg/mL (FIG. 16). The clinical trial results of the 58 test subjects were examined for the relationship between the survival time in the active-drug group and the survival time in the placebo group.

Among the 58 test subjects, 36 test subjects belonged to the active-drug group and showed the MST of the overall survival was 305 days. And 22 test subjects belonged to the placebo group and showed the MST of the overall survival was 191.5 days. The survival rate versus the number of days elapsed from the administration of the study product was compared between the two groups (FIG. 16). As a result, the active-drug group tended to be an extended overall survival (solid line) than the placebo group (broken line) (P=0.1239). The subgroup analysis (7) suggested that a combination of the two factors, namely the GM-CSF concentration of less than a predetermined threshold and the blood MCP-1 concentration within a predetermined range, could affect the survival time in the active-drug group as compared to that in the placebo group. A combination of the two factors is suggested as being able to be used as a prognostic factor for determining in advance the eligibility of a glioma patient for an active drug, and as being available as a prognostic factor for predicting the effect of the active drug on the glioma patient.

### [Subgroup Analysis (8)]

A subgroup analysis (8) was carried out to assess whether a combination of three factors, namely the blood MCP-1 concentration of less than a predetermined threshold, a high immunoreactivity to SART2 peptide, and the blood GM-CSF concentration, can determine in advance the eligibility of a test subject for an active drug. In the subgroup analysis (8), the clinical trial results of 9 test subjects in the active-drug group that showed the blood MCP-1 concentration of less than 100 pg/mL and for whom SART2 peptide was selected as a component of the active drug (SART2+) were examined for the relationship between the survival time and the blood GM-CSF concentration.

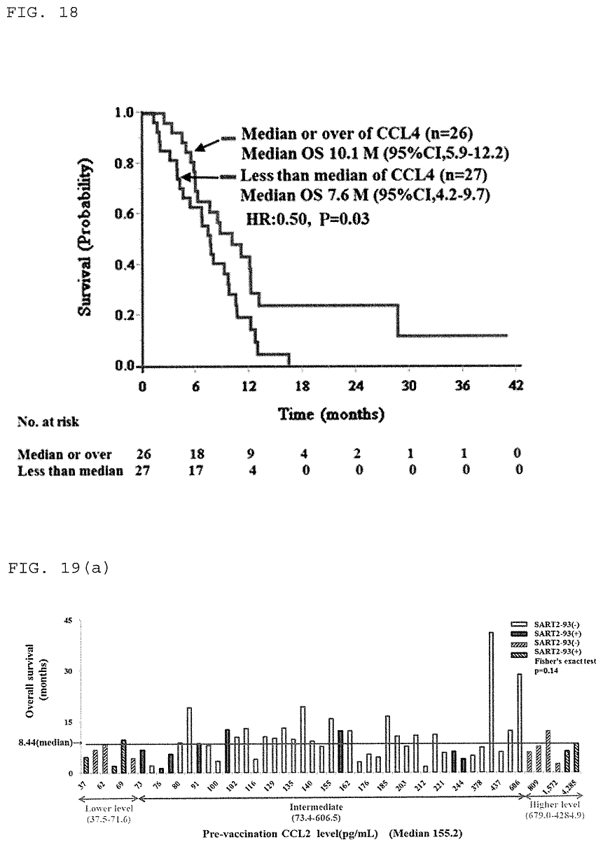

More specifically, there were 8 test subjects in the active-drug group who showed the blood MCP-1 concentration was less than 100 pg/mL and had the GM-CSF concentration of not less than 0.9 pg/mL (FIG. 14a, solid line). Among the 8 test subjects, 5 test subjects for whom SART2 peptide was selected as a component as an active drug (SART2+) showed the MST of the overall survival was 55 days. There were 7 test subjects who had the blood MCP-1 concentration of less than 100 pg/mL before the administration of the active drug and had the GM-CSF concentration of less than 0.9 pg/mL (FIG. 14a, broken line). Among the 7 test subjects, 4 test subjects for whom SART2 peptide was selected as a component as the active drug (SART2+) showed the MST of the overall survival was 246 days. The survival rate versus the number of days elapsed after the administration of the active drug was compared between the two groups (FIG. 17). As a result, the 5 test subjects in the former group tended to be shortened in overall survival (broken line) than the 4 test subjects in the latter group (solid line) (P=0.0527).

The subgroup analysis (8) revealed that a combination of the three factors, namely the blood MCP-1 concentration of a test subject before the administration of an active drug, the blood GM-CSF concentration, and the immunoreactivity to SART2 peptide, affects the effect (survival time) of the active drug on glioma patients. The combination of the three factors was suggested as being able to be used as a prognostic factor for determining in advance the eligibility of a test subject for an active drug.

Also, the subgroup analysis (8) revealed that a glioma patient shows the poorest prognosis in immunotherapy with the active drug when the glioma patient has the blood MCP-1 concentration of less than a predetermined threshold (for example, 100 pg/mL), a high immunoreactivity to SART2 peptide, and the blood GM-CSF concentration of not less than a predetermined threshold (for example, 0.9 pg/mL). A relationship between the poorest prognosis of the patient and a combination of three factors, namely MCP-1, GM-CSF, and SART2, is discussed below.

A clinical result's report of immunotherapy for breast cancer (Zia A. Dehqanzada, et al., Clin Cancer Res 2006; 12 (2)) is referred to. The report discloses that breast cancer patients having a high blood MCP-1 concentration before the administration of a HER2 peptide vaccine including GM-CSF had good prognosis. On the other hand, breast cancer patients having a low blood MCP-1 concentration before the administration of the vaccine had an increased blood MCP-1 concentration after the administration of the vaccine, resulting in a poor prognosis. When the report has taken into consideration, the poorest prognosis for the glioma patients who have a relative high blood level of a cytokine (for example, GM-CSF not less than 0.9 pg/mL) and a low MCP-1 concentration (for example, less than 100 pg/mL) is presumed to be caused by an increased MCP-1 concentration within the body of the patients after the administration of the peptide vaccine including SART2 peptide. Actually, the number of cases where the MCP-1 concentration increased was large in the active-drug group of patients to whom a peptide vaccine including SART2 peptide was administered (data not shown).

The prognoses for patients who had a high GM-CSF concentration before the administration of an active drug were also poor. In contrast, the number of cases where MCP-1 increased was small in the group of patients to whom a vaccine not including SART2 peptide was administered. A reason why an increased MCP-1 concentration causes a poor prognosis is presumed to be caused by inducing inhibitory T cells (Treg) via an MCP-1 receptor CCR4. Treg is known to suppress the effect of peptide vaccines (Chiara Vasco, et. al., J Neurooncol (2013) 115: 353-363; Justin T. Jordan, et al., Cancer Immunol Immunother (2008) 57: 123-131; and Xin Chen, et al., Int Immunopharmacol. 2016 May; 34: 244-9).

It is accordingly considered that when a glioma patient who has a relatively high blood concentration of a cytokine such as GM-CSF (for example, GM-CSF of not less than 0.9 pg/mL) and a low blood MCP-1 concentration (for example, less than 100 pg/mL) receives the administration of a peptide vaccine including SART2 peptide, the MCP-1 concentration increases within the patient's body (for example, blood concentration beyond 700 pg/mL), leading to the induction of Treg via MCP-1 receptor CCR4 expressed in Treg. As a result, the effect of the peptide vaccine is suppressed, leading to poor prognosis.

### [Subgroup Analysis (9)]

A subgroup analysis (9) was carried out to further find a prognostic factor by assessing the relationship between 34 soluble factors and the overall survival of 53 test subjects in the active-drug group from whom plasma samples before inoculation with a tailor-made peptide vaccine were available.

The 34 soluble factors included CCL-2 (MCP-1), GM-CSF, G-CSF, EGF, Eotaxin, FGF-basic, IFN-α, HGF, IFN-γ CXCL9 (MIG), VEGF, IL-1β, CCL3 (MIP-1α), CCL4 (MIP-1β), CCL5 (RANTES), IL-1RA, IL-2, IL-2r, IL-4, IL-5, IL-6, IL-7, IL-8, IL-10, IL-12 (p40/p70), IL-13, IL-15, IL-17, TNF-α, IP-(IFNγ-induced protein10; human CXCL10/IP-10; R&D Systems), BAFF (B-cell activating factor; human BAFF/BLyS/TNFSF13B; R&D Systems, Minneapolis, Minn.), TGFβ (human TGF-β1; R&D Systems), IL-21 and haptoglobin (Hp).

BAFF, TGFβ, IL-21, and Hp out of the soluble factors were measured by enzyme-linked immunosorbent assay (ELISA) (dual assay). The other thirty soluble factors than BAFF, TGFβ, IL-21, and Hp were measured by flowmetry (Luminex 200 system) using a fluorescent bead array (Human Cytokine 30-Plex Panel; Invitrogen, Carlsbad, Calif.). Measurement samples were obtained by thawing frozen samples of plasmas which were collected before inoculation with the active drug. ELISA was carried out with a commercially available kit such as human IL-21 ELISA Ready-SET-Go! (2nd Generation); eBioscience Inc., San Diego, Calif.).

The average value of duplicate samples was used for statistical analysis. The statistical analysis was carried out by student t test, chi-square test, Wilcoxon rank sum test, and Fisher's exact test in order to separately compare safety to a treatment, and quantitative and taxonomic variable in connection with immune response. Progression-free survival (PFS) and overall survival (OS) were analyzed by using all the test subjects randomly assigned to the active-drug group as a parent population. The PFS was calculated regarding the time from the date of random assignment to the date of non-censored observations (events) or to the final date of censored observation as the period. The OS was calculated regarding the time from the date of random assignment to the date of non-censored observation (event) or the final date of censored observation as the period. The time-to-event endpoint was analyzed by using the Kaplan-Meier method. A comparison of the PFS and the OS between treatments was made by using a log-rank test of a bilateral significance level of 5%. The hazard ratio (HR) and 95% confidence interval (CI) were calculated by using a Cox proportional hazard analysis.

### [Subgroup Analysis (9-1)]

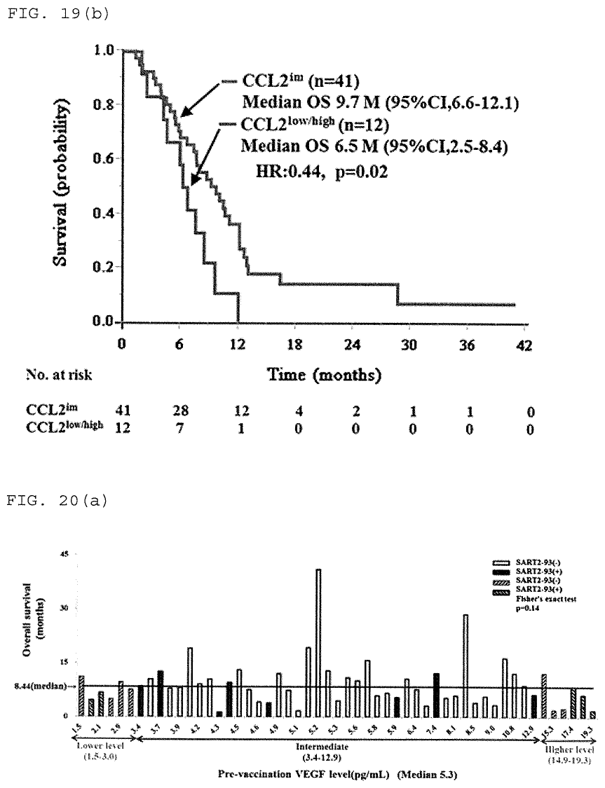

A significant correlation was found between the median value and the OS for CCL4 out of the 34 soluble factors examined (FIG. 18). The test subjects, from whom plasma samples included CCL4 of not less than the median value, showed the median survival time (MST) of the overall survival was 10.1 months (n=26), while the test subjects, from whom plasma samples included CCL4 of less than the median value, showed the MST of the overall survival was 7.6 months (n=27).

The subgroup analysis (9-1) revealed that the blood CCL4 concentration of a test subject before the administration of an active drug affects the survival time of the test subject to be treated with a peptide vaccine. This suggests that CCL4 can be used as a prognostic factor of determining in advance the eligibility of a glioma patient for an active drug.

### [Subgroup Analysis (9-2)]

A subgroup analysis (9-2) was carried out to further examine MCP-1 (CCL-2) for the availability as a prognostic factor that was suggested in the subgroup analysis (5).

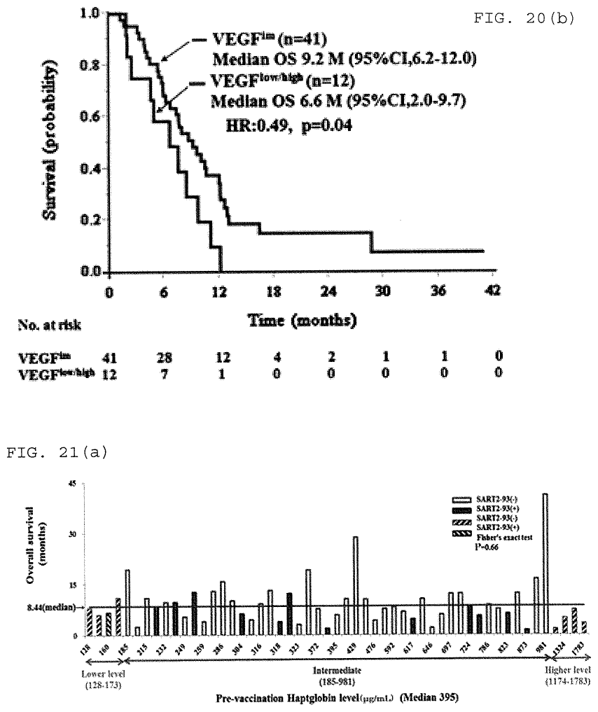

In the subgroup analysis (9-2), the blood CCL2 concentrations for plasma samples from 53 test subjects (glioma patients) were measured before the administration of the active drug (FIG. 19a). The 53 test subjects showed the median survival time (MST) of the overall survival was 8.44 months. Nine test subjects out of ten test subjects in the ascending order of the CCL2 level showed a shortened MST of overall survival compared to all the test subjects. Four test subjects out of six test subjects in descending order of the CCL2 level showed a shortened MST of overall survival compared to all the test subjects. These results suggest that a test subject having an extremely low or high CCL2 level (low/high) tends to be shortened in overall survival than a test subject having an intermediate CCL2 level (im).

In fact, the low/high-level CCL2 group including 12 test subjects (CCL2^{low/high}), which are the sum of 11% test subjects in ascending order of the blood CCL2 concentration (lower level; 6 test subjects) and 11% test subjects in descending order of the blood CCL2 concentration (higher level; 6 test subjects), showed the MST of 6.5 months, while the intermediate-level CCL2 group including the remaining 41 test subjects (CCL2^{im}) showed the MST of 9.7 months. The MST of CCL2^{im} was significantly long (P=0.02) (FIG. 19b).

The subgroup analysis (9-2) suggests that CCL2 can be used as a prognostic factor for determining in advance the eligibility of a glioma patient for an active drug.

### [Subgroup Analysis (9-3)]

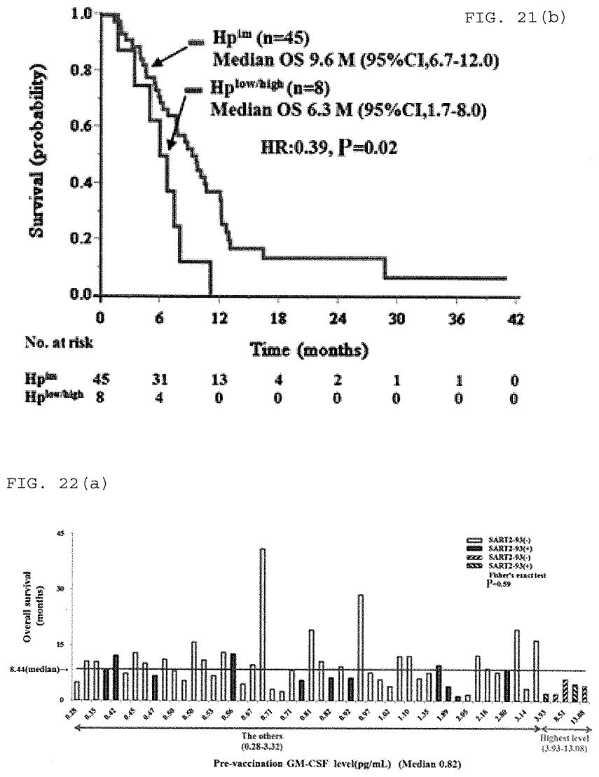

A subgroup analysis (9-3) examined blood VEGF concentrations of plasma samples from the 53 test subjects (glioma patients) before the administration of the active drug (FIG. 20a). The 53 test subjects showed the MST of 8.44 months. Four out of six test subjects in ascending order of the VEGF level showed a shortened MST of overall survival compared to all the test subjects. Five out of six test subjects in descending order of the VEGF level showed a shortened MST of overall survival compared to all the test subjects. These suggest that a test subject who has an extremely low or high VEGF level (low/high) tends to be shortened in overall survival than a test subject who has an intermediate VEGF level (im).

In fact, the low/high level VEGF group including 12 test subjects (VEGF^{low/high}), which are the sum of 11% test subjects in ascending order of the blood VEGF concentration (lower level; 6 test subjects) and 11% test subjects in descending order of the blood VEGF concentration (higher level; 6 test subjects), showed the MST of 6.6 months, while the intermediate-level VEGF group including the remaining 41 test subjects (VEGF^{im}) showed the MST of 9.2 months. The MST of VEGF^{im} was significantly long (P=0.04) (FIG. 20b).

The subgroup analysis (9-3) suggests that VEGF can be used as a prognostic factor for determining in advance the eligibility of a glioma patient for an active drug.

### [Subgroup Analysis (9-4)]

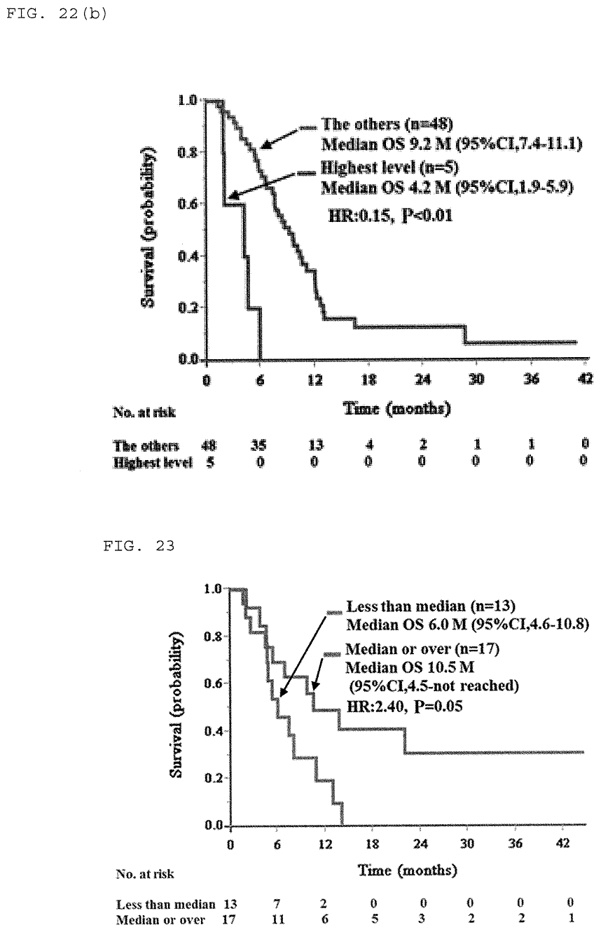

In a subgroup analysis (9-4), the blood haptoglobin (Hp) concentrations for plasma samples from the 53 test subjects (glioma patients) were measured before the administration of the active drug (FIG. 21a). The 53 test subjects showed the MST of 8.44 months. Three out of four test subjects in ascending order of the Hp level showed a shortened MST compared to all the test subjects. All four test subjects in descending order of the Hp level showed a shortened MST of overall survival compared to all the test subjects. These suggest that a test subject who has an extremely low or high Hp level (low/high) tends to be shortened in overall survival than a test subject who has an intermediate medium Hp level (im).

In fact, the low/high-level Hp group including 8 test subjects (Hp^{low/high}), which are the sum of 8% test subjects in ascending order of the blood Hp concentration (lower level; 4 test subjects) and 8% test subjects in descending order of the blood Hp concentration (higher level; 4 test subjects), showed the MST of 6.3 months, while the intermediate level Hp group including the remaining 45 test subjects (Hp^{im}) showed the MST of 9.6 months. The MST of HP^{im} was significantly long (P=0.02) (FIG. 21b).

The subgroup analysis (9-4) suggests that Hp can be used as a prognostic factor for determining in advance the eligibility of a glioma patient for an active drug.

### [Subgroup Analysis (9-5)]

IL-6 and IL-17 showed the same results as CCL2 and VEGF (data not shown). This suggests that IL-6 and IL-17 each can be used as a prognostic factor for determining in advance the eligibility of a glioma patient for an active drug. IL-7 also showed the same results as Hp (data not shown). This suggests that IL-7 can be used as a prognostic factor for determining in advance the eligibility of a glioma patient for an active drug.

### [Subgroup Analysis (9-6)]

The subgroup analyses (9-2) to (9-5) also assessed whether SART2 peptide, the availability of which as a prognostic factor was suggested in the subgroup analysis (2), was selected as a peptide vaccine (FIG. 19a, FIG. 20a and FIG. 21a). These assessments revealed that a group of test subjects, who have an intermediate level CCL2^{im}, VEGF^{im}, or IL-6^{im}, show a lower ratio of test subjects for whom SART2 peptide was selected as a peptide vaccine than its corresponding test subject group including the low/high-level test subjects.

### [Subgroup Analysis (9-7)]

A subgroup analysis (9-7) was carried out to further examine GM-CSF for the availability as a prognostic factor that was suggested in the subgroup analysis (3).

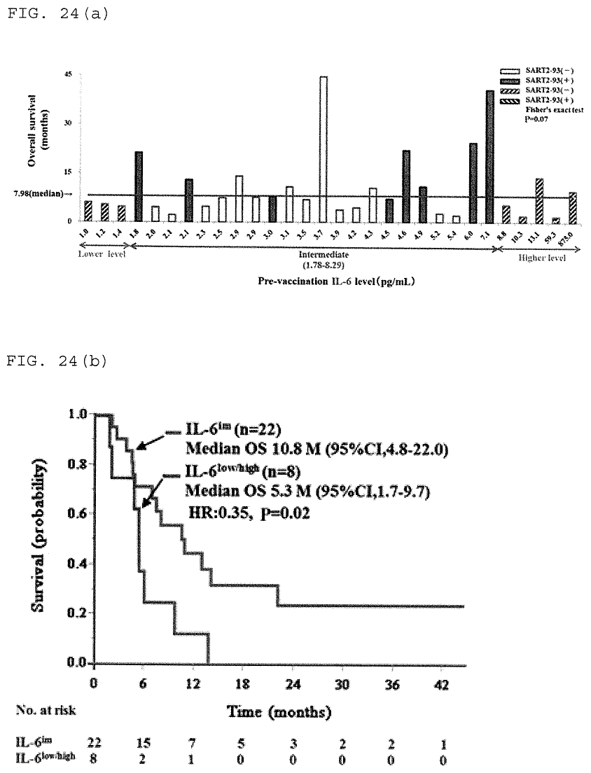

In the subgroup analysis (9-7), the blood GM-CSF concentrations in plasma samples from the 53 test subjects (glioma patients) were measured before the administration of the active drug (FIG. 22a). The 53 test subjects showed that the median survival time (MST) of overall survival was 8.44 months. All five test subjects in descending order of the GM-CSF level showed a shortened MST of overall survival compared to all the test subjects. This suggests that a test subject who has an extremely high GM-CSF level (high) tends to be shortened in overall survival than a test subject who has an intermediate GM-CSF level or less.

In fact, the high level GM-CSF group (GM-CSF^{high}) including 9% test subjects in descending order of the blood GM-CSF concentration (higher level; 5 test subjects) showed the MST of 4.2 months, while the intermediate or lower level GM-CSF group including the remaining 48 test subjects showed the MST of 9.2 months. The MST of the GM-CSF group of not more than medium level was significantly long (P<0.01) (FIG. 22b).

The subgroup analysis (9-7) further suggests that GM-CSF can be used as a prognostic factor for determining in advance the eligibility of a glioma patient for an active drug.

### [Subgroup Analysis (9-8)]

IL-1RA and an IL-10 showed the same results as GM-CSF (data not shown). This suggests that IL-1RA and IL-10 each can be used as a prognostic factor for determining in advance the eligibility of a glioma patient for an active drug.

The subgroup analyses (9-1) to (9-8) suggested the availabilities of 10 factors (CCL4, CCL2, VEGF, IL-6, IL-17, Hp, IL-7, GM-CSF, IL-1RA, and IL-10) as a prognostic factor. The subgroup analyses (9-2) to (9-8) suggested that a high-level CCL-2, VEGF, IL-6, IL-17, Hp, IL-7, GM-CSF, IL-1RA or IL-10 can be used as a prognostic factor.

### [Subgroup Analysis (9-9)]

The correlation coefficients between CCL2, VEGF, IL-6, IL-7, IL-17, and Hp, the availability of which as a prognostic factor was suggested in the subgroup analysis (9), were examined (Table 2).

TABLE 2
the correlation coefficient among CCL2, VEGF, IL-6, IL-7, IL-17, and Hp.

| Cytokines | CCL2 r(p) | | VEGF r(p) | | IL-6 r(p) | | IL-7 r(p) | | IL-17 r(p) | | Haptglobin r(p) | |
|---|---|---|---|---|---|---|---|---|---|---|---|---|
| CCL2 | − | (−) | 0.73 | (<0.01) | 0.71 | (<0.01) | 0.15 | (0.19) | 0.34 | (<0.01) | 0.00 | (0.97) |
| VEGF | 0.73 | (<0.01) | − | (−) | 0.91 | (<0.01) | 0.34 | (<0.01) | 0.50 | (<0.01) | 0.06 | (0.58) |
| IL-6 | 0.71 | (<0.01) | 0.91 | (<0.01) | − | (−) | 0.26 | (0.02) | 0.48 | (<0.01) | 0.04 | (0.71) |
| IL-7 | 0.15 | (0.19) | 0.34 | (<0.01) | 0.26 | (0.02) | − | (−) | 0.38 | (<0.01) | −0.09 | (0.41) |
| IL-17 | 0.34 | (<0.01) | 0.50 | (<0.01) | 0.48 | (<0.01) | 0.3S | (<0.01) | − | (−) | 0.18 | (0.10) |
| Haptglobin | 0.00 | (0.97) | 0.06 | (0.58) | 0.04 | (0.71) | −0.09 | (0.41) | 0.18 | (0.10) | − | (−) |

r: correlation coefficient;
p: probability

High correlations were found between CCL2, VEGF, and IL-6 (r>0.7). This suggests that CCL2, VEGF, and IL-6 mutually relate to each other in determining in advance the eligibility of a glioma patient for an active drug. In the case where not less than two prognostic factors are used in determining in advance the eligibility of a glioma patient for a peptide vaccine treatment, when CCL2 is used as a prognostic factor, the mutual relationship suggests that a prognostic factor would be beneficially chosen from other prognostic factors than VEGF and IL-6 with which CCL2 shows a high correlation.

Relatively high correlations were found between IL-17 and CCL2, VEGF or IL-6 (0.4<r<0.5). This suggests that IL-17, CCL2, VEGF, and IL-6 have mutual relationships in determining in advance the eligibility of a glioma patient for an active drug.

### [Subgroup Analysis (10)]

A subgroup analysis (10) was carried out to assess a correlation between the 34 soluble factors described above and the overall survival of 30 test subjects, plasma samples of which in the placebo group before the administration of the placebo were available.

### [Subgroup Analysis (10-1)]

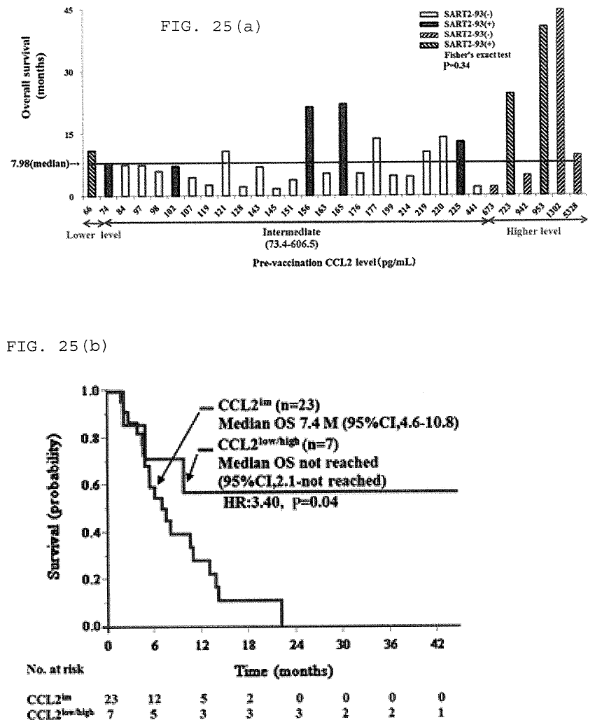

IL-15 among the 34 soluble factors examined showed a significant correlation between the median and the OS (FIG. 23). The test subjects, who had plasma samples including IL-15 of not less than the median, showed the median survival time (MST) of the overall survival of 10.5 months (n=17), while the test subjects, who had plasma samples including IL-15 of less than the median, showed the MST of 6.0 months (n=13).

The subgroup analysis (10-1) revealed that the blood IL-15 concentration of a test subject affects its overall survival. This suggests that an IL-15 can be used as a biomarker for predicting the overall survival of a glioma patient.

### [Subgroup Analysis (10-2)]

A subgroup analysis (10-2) was carried out to further examine IL-6 for the availability as a prognostic factor that was suggested in the subgroup analysis (9-5).

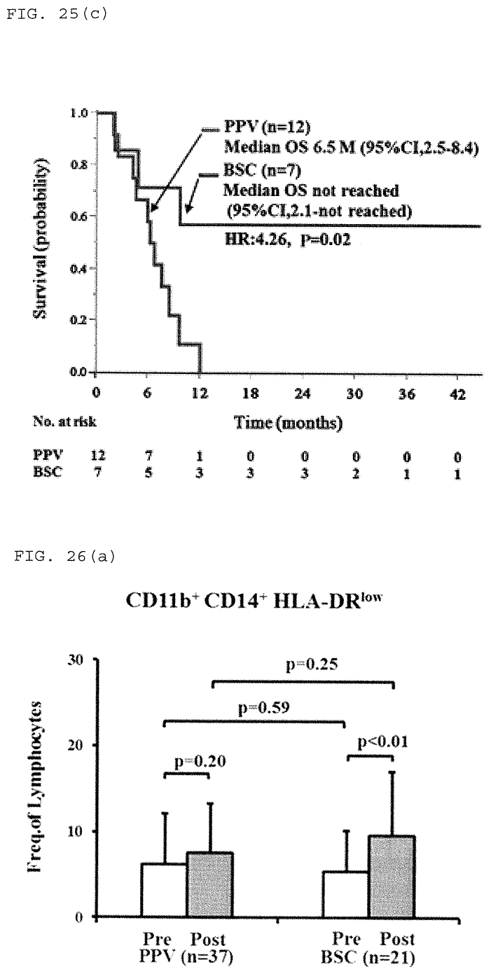

In the subgroup analysis (10-2), blood IL-6 concentrations in plasma samples from the 30 test subjects (glioma patients) were measured before the administration of the placebo (FIG. 24a). The 30 test subjects showed the median overall survival time (MST) of 7.98 months. Three test subjects in ascending order of the IL-6 level showed a shortened overall survival than the MST of all the test subjects. Three out of five test subjects in descending order of the IL-6 level showed a shortened overall survival than the MST of all the test subjects. This suggests that a test subject who has an extremely low or high IL-6 level (low/high) tends to be shortened in overall survival than a test subject having an intermediate IL-6 level (im).

In fact, the low/high level IL-6 group including 8 test subjects (IL-6^{low/high}), which are the sum of 10% test subjects in ascending order of the blood IL-6 concentration (lower level; 3 test subjects) and 17% test subjects in descending order of the blood IL-6 concentration (higher level; 5 test subjects), showed the MST of 5.3 months, while the intermediate level CCL2 group including the remaining 22 test subjects (CCL2^{im}) showed the MST of 10.8 months. The MST of IL-6^{im} was significantly long (P=0.02) (FIG. 24b).

The subgroup analysis (10-2) revealed that the blood IL-6 concentration of a test subject affects its overall survival. This suggests that IL-6 can be used as a biomarker for predicting the overall survival of a glioma patient.

### [Subgroup Analysis (10-3)]

A subgroup analysis (10-3) was carried out to further examine CCL-2 for the availability as a prognostic factor that was suggested in the subgroup analyses (5) and (9-2).

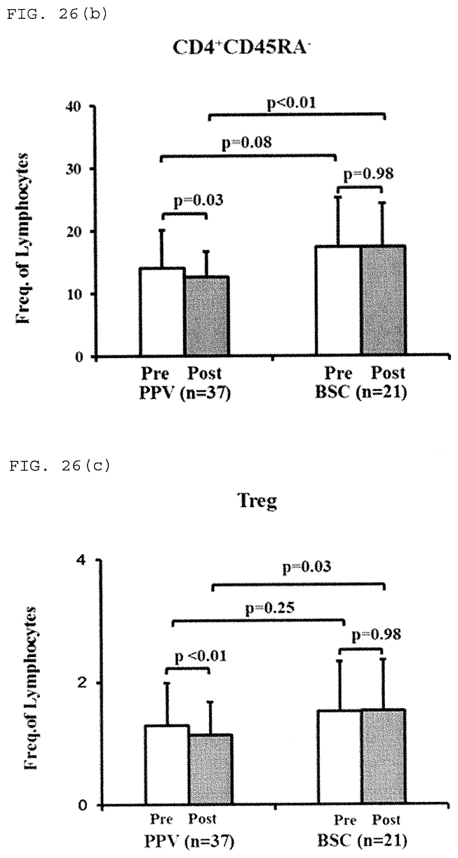

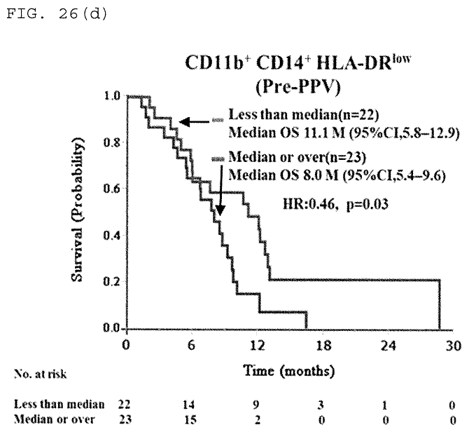

In the subgroup analysis (10-3), the blood CCL-2 concentrations for plasma samples from the 30 test subjects (glioma patients) were measured before the administration of the active drug (FIG. 25a). The 30 test subjects showed the MST of 7.98 months. One test subject in the lowest of the CCL-2 level showed an extended overall survival than the MST of all the test subjects. In addition, four out of six test subjects in descending order of the CCL-2 level showed an extended overall survival than the MST of all the test subjects. This suggests that a test subject who has an extremely low or high CCL-2 level (low/high) tends to be extended in overall survival than a test subject who has an intermediate CCL-2 level (im).

In fact, the low/high level CCL-2 group including 7 test subjects (CCL-2^{low/high}), which are the sum of 3% test subjects in ascending order of the blood CCL-2 concentration (lower level; single test subject) and 20% test subjects in descending order of the blood CCL-2 concentration (higher level; 6 test subjects), showed the MST was “unreachable”, while the intermediate level CCL2 group (CCL^{im}) including the remaining 23 test subjects showed the MST was 7.4 months. The MST of CCL2im was significantly short (P=0.04) (FIG. 25b).

The subgroup analysis (10-3) revealed that the blood CCL2 concentration of a test subject affects its overall survival. This suggests that CCL2 can be used as a biomarker for predicting the overall survival of a glioma patient.

### [Subgroup Analysis (10-4)]

A subgroup analysis (10-4) was carried out to examine CCL2 for the availability as a biomarker for predicting the overall survival of a glioma patient that was suggested in the subgroup analysis (10-3).

The subgroup analysis (10-3) suggested that the overall survival of low/high-level CCL2 group (CCL2^{low/high}) tends to be extended. This suggests that the overall survival of a subject having CCL2^{low/high} would be shortened when the subject is inoculated with a tailor-made peptide vaccine (personalized peptide vaccine; PPV). In fact, for 19 test subjects in a low/high-level CCL group (CCL2^{low/high}) 12 test subjects in an active-drug group showed the MST was 6.5 months, which was significantly shortened than the MST (unreachable) of 7 test subjects in the placebo group (P=0.02).

The subgroup analysis (10-4) revealed that the blood CCL2 concentration of a test subject before the administration of an active drug affects its survival time in an active-drug group compared to the placebo group. This suggests that CCL2 can be used as a prognostic factor for predicting in advance the effect of an active drug on a glioma patient.

### [Subgroup Analysis (10-5)]

The MSTs of VEGF^{low/high}, IL-7^{low/high}, and IL-17^{low/high} in the active-drug group were compared to those in the placebo group, similar to the subgroup analysis (10-4). The comparison revealed that any significant difference was not found between the active-drug group and the placebo group (data not shown).

### [Subgroup Analysis (11)]

A subgroup analysis (11) was carried out to find a further prognostic factor by assessing the correlation between a T cell subset and the overall survival of 58 test subjects, from which the peripheral blood mononuclear cell data before and after inoculation with the active drug or the placebo were available. Among the 58 test subjects, 37 test subjects belonged to the active-drug group, and 21 test subjects belonged to the placebo group.

Peripheral blood monocytes (PBMC) were stained with an anti-CD3 antibody, anti-CD4 antibody, anti-CD8 antibody, and anti-CD45RA antibody to analyze T cell subsets. Treg cell analysis was carried out by using True-Nuclear One Step Staining Human Treg Flow Kit (Biolegend, San Diego, Calif.), PBMCs were stained with an anti-CD4 antibody, anti-CD25 antibody, anti-CD45RA antibody, and anti-FoxP3 antibody. Myeloid-derived suppressor cells (MDSC) analysis was carried out by staining PBMCs (0.5×10⁶ cells) with a monoclonal anti-CD3 antibody, anti-CD11b antibody, anti-CD14 antibody, anti-CD16 antibody, anti-CD19 antibody, anti-CD33 antibody, anti-CD56 antibody, and anti-HLA-DR antibody (all were obtained from Biolegend).

A lineage (Lin) marker was identified as being positive to CD3, CD16, CD19, and CD56. Lin negative cells were identified as CD3⁻, CD19⁻, CD56⁻, and CD14⁻. Granulocytic MDSCs were identified as being positive to CD33, CD15, and CD11b. Monocytes were identified as positive to CD14 and CD11b. Monocyte MDSCs (m-MDSC) were identified as Lin⁻CD11b⁺ and CD14HLA-DR^{low/-}, based on the publicly known literature (Cell Rep. 2016; 17 (12): 3219-3232 and Cancer Immunology Research. 2014; 2 (8): 812-821).

Immunosuppressive monocytes were identified as being positive to CD11b⁺CD14⁺HLA-DR^{low/-} based on the publicly known literature (PLoS One. 2015; 10 (5): e0126022). Treg and e-Treg were identified respectively as CD4⁺CD25⁺FoxP3⁺T cells and CD4⁺CD25⁺CD45RA−FoxP3⁺T cells (Clin Cancer Res. 2016; 22 (12): 2908-2918, Cell Rep. 2016; 17 (12): 3219-3232, Nat Med. 2016; 22 (6): 679-686). Treg and MDSC were analyzed by using FLOW JO ver7.6.5 (FLOWJO, Ashland, Oreg.).

### [Subgroup Analysis (11-1)]

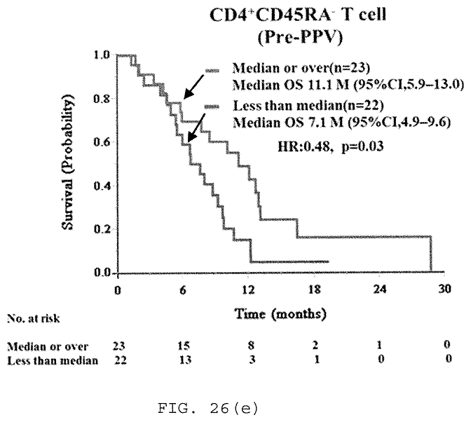

Significant differences were not found in 37 test subjects who were inoculated with the tailor-made peptide vaccine (personalized a peptide vaccine; PPV) (active-drug group) for the ratio of CD11b⁺CD14⁺HLA-DR^{low} immunosuppressive monocytes between before and after the inoculation with the peptide vaccine (FIG. 26a; P=0.20). A significant increase in the number of these cells was found for 21 test subjects who received a Best Supportive Care (BSC) after the inoculation of the placebo (placebo group) (FIG. 26a; p<0.01).

Significant decreases were found in the number of CD3⁺CD4⁺CD45RA⁻T cells that had activation/memory T helper phenotypes (FIG. 26b; P=0.03), CD3⁺CD8′CD45RA⁻T cells that had activation/memory CTL phenotype (data not shown), and CD4⁺CD25⁺FoxP3⁺ cells (Treg) (FIG. 26c; p<0.01) for the test subjects in the active-drug group after the inoculation with the active drug. This suggests that the active drug enabled these activated T cells to migrate from blood circulation to a tumor site. In contrast, no significant difference was found in the number of other T cell subsets such as Lin⁻CD11b⁺CD14⁺HLA-DR^{-/low} m-MDSC and granulocytic MDSC for the test subjects in the active-drug group before and after the inoculation with the peptide vaccine (data not shown).

### [Subgroup Analysis (11-2)]

Next, 45 test subjects in the active-drug group were examined for the correlation between the ratio of CD11b⁺CD14⁺HLA-DR^{low} immunosuppressive monocytes and the overall survival of the test subjects (FIG. 26d). The test subjects who had a low-ratio CD11b⁺CD14⁺HLA-DR^{low} immunosuppressive monocytes (less than the median) showed the MST of 11.1 months (n=22), which was significantly longer than the MST (8.0 months) of the test subjects who had a high ratio CD11b⁺CD14⁺HLA-DR^{low} immunosuppressive monocytes (not less than the median) (n=23) (P=0.03).

The subgroup analysis (11-2) revealed that the ratio of T cell subset CD11b⁺CD14⁺HLA-DR^{low} immunosuppressive monocytes in a test subject after the administration of an active drug correlates with the survival time of the test subject treated with the peptide vaccine. This suggests that CD11b⁺CD14⁺HLA-DR^{low} immunosuppressive monocytes can be used as a factor for determining the eligibility of a glioma patient for an active drug after the administration.

### [Subgroup Analysis (11-3)]

The 45 test subjects in the active-drug group were examined for the correlation between the ratio of CD11b⁺CD14⁺HLA-DR⁻ immunosuppressive monocytes and the overall survival of the test subjects (data not shown). The obtained results were similar to those in the subgroup analysis (11-2) (data not shown). This suggests that CD11b⁺CD14⁺HLA-DR⁻ immunosuppressive monocytes can be used as a factor for determining the eligibility of a glioma patient for an active drug after the administration.

### [Subgroup Analysis (11-4)]

The 45 test subjects in the active-drug group were examined for the correlation between the ratio of CD3⁺CD4⁺CD45RA⁻T cells and the overall survival of the test subjects (FIG. 26e). On the contrary to the subgroup analyses (11-2) and (11-3), the test subjects who had a high ratio CD3⁺CD4⁺CD45RA⁻T cells (not less than the median) showed the MST of 11.1 months (n=23), which was significantly longer than the MST (7.1 months) of the test subjects who had a low ratio CD3⁺CD4⁺CD45RA⁻T cells (less than the median) (n=22) (P=0.03).

The subgroup analysis (11-4) revealed that the ratio of T cell subset CD3⁺CD4⁺CD45RA⁻T cells in a test subject after the administration of an active drug correlates with the survival time of the test subject to be treated with the peptide vaccine. This suggests that CD3⁺CD4⁺CD45RA⁻T cells can be used as a factor for determining the eligibility of a glioma patient for an active drug after the administration.

### [Subgroup Analysis (11-5)]

The 45 test subjects in the active-drug group were examined for the correlation between the ratio of the following T cell subset and the overall survival of the test subjects (data not shown). No correlation was found between the ratio of these T cell subset and the overall survival of the test subjects in this analysis.

Lin⁻CD11b⁺CD14⁺HLA-DR^{-/low}m-MDSC, granulocytic MDSC, CD3⁺CD8⁺CD45RA⁻T cells, CD4⁺CD25⁺T cells, CD4⁺Foxp3⁺Treg or CD4⁺CD25⁺CD45RA−FoxP3⁺ effector Treg (e-Treg).

### [Subgroup Analysis (12)]

A subgroup analysis (12) was carried out to examine a correlation for the overall survival of 29 test subjects in the placebo group, the data of which were available for the peripheral blood mononuclear cells before the inoculation of the placebo. The test subjects who had a low ratio of CD11b⁺CD14⁺HLA-DR⁻ immunosuppressive monocytes (less than the median) showed the MST of 13.7 months, which was longer than the MST (7.4 months) of test subjects who had a high ratio of CD11b⁺CD14⁺HLA-DR⁻ immunosuppressive monocytes (not less than the median) (P=0.07) (data not shown). No correlation was found between the ratio of the immune cell subset examined and the overall survival of the test subjects (data not shown).

1. A method for determining whether a subject suffering from a brain tumor is an eligible person for a peptide vaccine composition including at least one peptide antigen, comprising:
evaluating a risk of the subject with respect to the peptide vaccine composition to obtain an evaluation; and
determining whether the subject is an eligible person for the peptide vaccine composition based on the evaluation to obtain a determination,
wherein obtaining the evaluation comprises using at least one prognostic factor selected from the group consisting of GM-CSF, at least one SART2, MCP-1, VEGF, IL-6, IL-7, IL-10, IL-17, IL-1RA, CCL4, and haptoglobin.

2. The determination method according to claim 1, wherein the at least one prognostic factor includes at least one selected from GM-CSF, the at least one SART2, and MCP-1.

3. The determination method according to claim 1, wherein the at least one prognostic factor includes at least two selected from the group.

4. The determination method according to claim 1, wherein obtaining the evaluation comprises
evaluating a risk of the subject with respect to the peptide vaccine composition by comparing a level of granulocyte-macrophage colony-stimulating factor (GM-CSF) in a blood sample from the subject with a GM-CSF threshold to obtain an evaluation A; and further comprises
either one or both of evaluating a risk of the subject with respect to the peptide vaccine composition by comparing an immunoreactivity of the subject to the at least one SART2 peptide with a SART2 threshold to obtain an evaluation B and evaluating a risk of the subject with respect to the peptide vaccine composition by comparing a level of Monocyte Chemoattractant Protein-1 (MCP-1) in a blood sample from the subject with a MCP-1 threshold to obtain an evaluation C;
wherein the evaluation A is given as “having no risk” when the GM-CSF level is less than the GM-CSF threshold, or is given as “having a risk” when the GM-CSF level is not less than the GM-CSF threshold,
the evaluation B is given as “having no risk” when the immunoreactivities of the subject to both of the at least one SART2 peptide are less than the SART2 threshold, or is given as “having a risk” when the immunoreactivity of the subject to any one of the at least one SART2 peptide is not less than the SART2 threshold, and
in the case where the MCP-1 threshold includes an MCP-1 threshold (1), the evaluation C is given as “having a risk” when the MPC-1 level is less than the MCP-1 threshold (1); or in the case where the MCP-1 threshold includes an MCP-1 threshold (1) and an MCP-1 threshold (2) that is greater than the value of the MCP-1 threshold (1), the evaluation C is given as “having no risk” when the MCP-1 level is not less than the MCP-1 threshold (1) and is less than the MCP-1 threshold (2); or is given as “having a risk” when the MCP-1 level is less than the MCP-1 threshold (1) or not less than the MCP-1 threshold (2).

5. The determination method according to claim 4, wherein the determination is given that the subject is an “eligible person” for the peptide vaccine composition,
when obtaining the evaluation comprises obtaining the evaluation A and obtaining the evaluation B, and either one or both of the evaluation A and the evaluation B are given as “having no risk”; or
when obtaining the evaluation comprises obtaining the evaluation A and obtaining the evaluation C, and either one or both of the evaluation A and the evaluation C are given as “having no risk”.

6. The determination method according to claim 4, wherein the determination is given that the subject is an “ineligible person” for the peptide vaccine composition,
when obtaining the evaluation comprises obtaining the evaluation A and obtaining the evaluation B, and both of the evaluation A and the evaluation B are given as “having a risk”;
when obtaining the evaluation comprises obtaining the evaluation A and obtaining the evaluation C, and both of the evaluation A and the evaluation C are given as “having a risk”; or
when obtaining the evaluation comprises obtaining the evaluation A, obtaining the evaluation B, and obtaining the evaluation C, and the evaluation A, the evaluation B, and the evaluation C are all given as “having a risk”.

7. The determination method according to claim 5, wherein the at least one SART2 peptide includes either one or both of SART2-93 peptide (SEQ ID NO:1) and SART2-161 peptide (SEQ ID NO:9).

8. The determination method according to claim 1, wherein
the subject is HLA-A24 positive;
the peptide vaccine composition includes at least two peptide antigens selected from the peptide antigen group containing SART2-93 peptide (SEQ ID NO:1), SART3-109 peptide (SEQ ID NO:2), Lck-208 peptide (SEQ ID NO:3), PAP-213 peptide (SEQ ID NO:4), PSA-248 peptide (SEQ ID NO:5), EGF-R-800 peptide (SEQ ID NO:6), MRP3-503 peptide (SEQ ID NO:7), MRP3-1293 peptide (SEQ ID NO:8), SART2-161 peptide (SEQ ID NO:9), Lck-486 peptide (SEQ ID NO:10), Lck-488 peptide (SEQ ID NO:11), PSMA-624 peptide (SEQ ID NO:12), EZH2-735 peptide (SEQ ID NO:13) and PTHrP-102 peptide (SEQ ID NO:14), and
the at least two peptide antigens are selected in the descending order of immunoreactivities of the subject to the peptide antigens.

9. The determination method according to claim 1, wherein the brain tumor is glial tumor or glioma.

10. The determination method according to claim 9, wherein the brain tumor is glioma, and the glioma is resistant to a temozolomide therapy.

11. The determination method according to claim 1, wherein the peptide vaccine composition includes at most four peptide antigens.

12. The determination method according to claim 1, further comprising:
evaluating a risk with respect to administration of the peptide vaccine composition based on a lymphocyte in a blood sample from the subject to obtain a risk; and
determining whether the administration of the peptide vaccine composition is canceled or not based on the risk,
wherein the lymphocyte is at least one selected from the group consisting of a CD11b+CD14+HLA-DRlow immunosuppressive monocyte, a CD3+CD4+CD45RA−T cell and a CD4+CD25+FoxP3+ cell (Treg).

13. A method for treating a subject suffering from a brain tumor by administering a peptide vaccine composition including at least one peptide antigen, wherein the subject is a person who is determined as an eligible person for the peptide vaccine composition based on an evaluation on a risk of the subject with regard to the peptide vaccine composition; and
the evaluation is made based on at least one prognostic factor selected from the group consisting of GM-CSF, at least one SART2, MCP-1, VEGF, IL-6, IL-7, IL-10, IL-17, IL-1RA, CCL4, and haptoglobin.

14. The treatment method according to claim 13, wherein the peptide vaccine composition includes at least two peptide antigens selected from the peptide antigen group containing SART2-93 peptide (SEQ ID NO:1), SART3-109 peptide (SEQ ID NO:2), Lck-208 peptide (SEQ ID NO:3), PAP-213 peptide (SEQ ID NO:4), PSA-248 peptide (SEQ ID NO:5), EGF-R-800 peptide (SEQ ID NO:6), MRP3-503 peptide (SEQ ID NO:7), MRP3-1293 peptide (SEQ ID NO:8), SART2-161 peptide (SEQ ID NO:9), Lck-486 peptide (SEQ ID NO:10), Lck-488 peptide (SEQ ID NO:11), PSMA-624 peptide (SEQ ID NO:12), EZH2-735 peptide (SEQ ID NO:13), and PTHrP-102 peptide (SEQ ID NO:14).

15. A kit for use in the determination method according to claim 1, containing a reagent for measuring at least one prognostic factor selected from the group consisting of GM-CSF, at least one SART2, MCP-1, VEGF, IL-6, IL-7, IL-10, IL-17, IL-1RA, CCL4, and haptoglobin.

16. The determination method according to claim 6, wherein the at least one SART2 peptide includes either one or both of SART2-93 peptide (SEQ ID NO:1) and SART2-161 peptide (SEQ ID NO:9).

17. The treatment method according to claim 13, wherein the brain tumor is glioma and the glioma is resistant to a temozolomide therapy.


SEQUENCE LISTING (14 sequences)

SEQ ID NO: 1 - protein, 9 aa, Artificial Sequence
  SART2 (amino acid position 93-101)
DYSARWNEI

SEQ ID NO: 2 - protein, 10 aa, Artificial Sequence
  SART3 (amino acid position 109-118)
VYDYNCHVDL

SEQ ID NO: 3 - protein, 9 aa, Artificial Sequence
  p56 lck (amino acid position 208-216)
HYTNASDGL

SEQ ID NO: 4 - protein, 9 aa, Artificial Sequence
  PAP (amino acid position 213-221)
LYCESVHNF

SEQ ID NO: 5 - protein, 10 aa, Artificial Sequence
  PSA (amino acid position 248-257)
HYRKWIKDTI

SEQ ID NO: 6 - protein, 10 aa, Artificial Sequence
  EGF-R (amino acid position 800-809)
DYVREHKDNI

SEQ ID NO: 7 - protein, 9 aa, Artificial Sequence
  MRP3 (amino acid position 503-511)
LYAWEPSFL

SEQ ID NO: 8 - protein, 10 aa, Artificial Sequence
  MRP3 (amino acid position 1293-1302)
NYSVRYRPGL

SEQ ID NO: 9 - protein, 9 aa, Artificial Sequence
  SART2 (amino acid position 161-169)
AYDFLYNYL

SEQ ID NO: 10 - protein, 9 aa, Artificial Sequence
  p56 lck (amino acid position 486-494)
TFDYLRSVL

SEQ ID NO: 11 - protein, 10 aa, Artificial Sequence
  p56 lck (amino acid position 488-497)
DYLRSVLEDF

SEQ ID NO: 12 - protein, 9 aa, Artificial Sequence
  PSMA (amino acid position 624-632)
TYSVSFDSL

SEQ ID NO: 13 - protein, 9 aa, Artificial Sequence
  EZH2 (amino acid position 735-743)
KYVGIEREM

SEQ ID NO: 14 - protein, 10 aa, Artificial Sequence
  PTHrP (amino acid position 102-111)
RYLTQETNKV